SEQUENCE LISTING (19 sequences)

SEQ ID NO: 1 - DNA, 1401 bp, Homo sapiens
  misc_feature (286)..(1680): Human liver Glucokinase 2 cDNA
atggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtga

SEQ ID NO: 2 - protein, 466 aa, Homo sapiens
MAMDVTRSQAQTALTLVEQILAEFQLQEEDLKKVMRRMQKEMDRGLRLETHEEASVKMLPTYVRSTPEGSEVGDFLSLDLGGTNFRVMLVKVGEGEEGQWSVKTKHQMYSIPEDAMTGTAEMLFDYISECISDFLDKHQMKHKKLPLGFTFSFPVRHEDIDKGILLNWTKGFKASGAEGNNVVGLLRDAIKRRGDFEMDVVAMVNDTVATMISCYYEDHQCEVGMIVGTGCNACYMEEMQNVELVEGDEGRMCVNTEWGAFGDSGELDEFLLEYDRLVDESSANPGQQLYEKLIGGKYMGELVRLVLLRLVDENLLFHGEASEQLRTRGAFETRFVSQVESDTGDRKQIYNILSTLGLRPSTTDCDIVRRACESVSTRAAHMCSAGLAGVINRMRESRSEDVMRITVGVDGSVYKLHPSFKERFHASVRRLTPSCEITFIESEEGSGRGAALVSAVACKKACMLGQ

SEQ ID NO: 3 - DNA, 28 bp, Artificial Sequence
  Description of Artificial Sequence LGK-2 Primer
ccggatccagatggcgatggatgtcaca

SEQ ID NO: 4 - DNA, 22 bp, Artificial Sequence
  Description of Artificial Sequence SAC Ib Primer
ggtttgcagagctctcgtccac

SEQ ID NO: 5 - DNA, 22 bp, Artificial Sequence
  Description of Artificial Sequence SAC Ia Primer
gtggacgagagctctgcaaacc

SEQ ID NO: 6 - DNA, 25 bp, Artificial Sequence
  Description of Artificial Sequence GLK-3 Primer
ctgaattcactggcccagcatacag

SEQ ID NO: 7 - DNA, 26 bp, Artificial Sequence
  Description of Artificial Sequence LGK-3 Primer
ccctcgagatggcgatggatgtcaca

SEQ ID NO: 8 - DNA, 25 bp, Artificial Sequence
  Description of Artificial Sequence GLK-3-2 Primer
ctaagcttactggcccagcatacag

SEQ ID NO: 9 - DNA, 21 bp, Artificial Sequence
  Description of Artificial Sequence Ser336Val Primer A
tcgtggtccaggtggagagcg

SEQ ID NO: 10 - DNA, 21 bp, Artificial Sequence
  Description of Artificial Sequence Ser336Val Primer B
cgctctccacctggaccacga

SEQ ID NO: 11 - DNA, 21 bp, Artificial Sequence
  Description of Artificial Sequence Ser336Leu Primer A
tcgtgctgcaggtggagagcg

SEQ ID NO: 12 - DNA, 21 bp, Artificial Sequence
  Description of Artificial Sequence Ser336Leu Primer B
cgctctccacctgcagcacga

SEQ ID NO: 13 - DNA, 21 bp, Artificial Sequence
  Description of Artificial Sequence Ser336Ile Primer A
tcgtgattcaggtggagagcg

SEQ ID NO: 14 - DNA, 21 bp, Artificial Sequence
  Description of Artificial Sequence Ser336Ile Primer B
cgctctccacctgaatcacga

SEQ ID NO: 15 - DNA, 22 bp, Artificial Sequence
  Description of Artificial Sequence Asp205Ala Primer A
ggtgaatgcaacggtggccacg

SEQ ID NO: 16 - DNA, 23 bp, Artificial Sequence
  Description of Artificial Sequence Asp205Ala Primer B
cgtggccaccgttgcattcaccc

SEQ ID NO: 17 - DNA, 22 bp, Artificial Sequence
  Description of Artificial Sequence Primer 4A
gacttcctggacaagcatcaga

SEQ ID NO: 18 - protein, 464 aa, Homo sapiens
  MISC_FEATURE: Human liver Glucokinase
MPRPRSQLPQPNSQVEQILAEFQLQEEDLKKVMRRMQKEMDRGLRLETHEEASVKMLPTYVRSTPEGSEVGDFLSLDLGGTNFRVMLVKVGEGEEGQWSVKTKHQMYSIPEDAMTGTAEMLFDYISECISDFLDKHQMKHKKLPLGFTFSFPVRHEDIDKGILLNWTKGFKASGAEGNNVVGLLRDAIKRRGDFEMDVVAMVNDTVATMISCYYEDHQCEVGMIVGTGCNACYMEEMQNVELVEGDEGRMCVNTEWGAFGDSGELDEFLLEYDRLVDESSANPGQQLYEKLIGGKYMGELVRLVLLRLVDENLLFHGEASEQLRTRGAFETRFVSQVESDTGDRKQIYNILSTLGLRPSTTDCDIVRRACESVSTRAAHMCSAGLAGVINRMRESRSEDVMRITVGVDGSVYKLHPSFKERFHASVRRLTPSCEITFIESEEGSGRGAALVSAVACKKACMLGQ

SEQ ID NO: 19 - protein, 465 aa, Homo sapiens [sequence omitted]

(19) United States
(12) Patent Application Publication
(10) Pub. No.: US 2007/0219346 A1
(43) Pub. Date: Sep. 20, 2007

(54) Glucose sensor and uses thereof

(75) Inventor: Mark A. Trifiro, Montreal (CA)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: McGill University, Montreal (CA)

(21) Appl. No.: 11/605,173

(22) Filed: Nov. 27, 2006

Related U.S. Application Data
(63) Continuation-in-part of application No. 10/421,360, filed on Apr. 22, 2003, now abandoned.
(60) Provisional application No. 60/374,126, filed on Apr. 22, 2002.

(51) Int. Cl. C07K 14/605

(52) U.S. Cl. 530/308

(57) ABSTRACT

The present invention provides a glucokinase protein in which the catalytic activity has been disabled in order to enable its use as a glucose sensor. The catalytically disabled glucokinase protein can be used as the glucose sensor in hand-held glucose monitors and in implantable glucose monitoring devices. The glucose sensor can also be incorporated into biomedical devices for the continuous monitoring of glucose and administration of insulin.

### RELATED US APPLICATION DATA

This application is a Continuation-in-Part of application Ser. No. 10/421,360 filed on Apr. 22, 2003.

### FIELD OF THE INVENTION

The present invention pertains to the field of glucose sensors, in particular, to a glucokinase protein, wherein the catalytic enzymatic activity has been disabled, yet the protein retains a high specific affinity for and ability to bind glucose.

### BACKGROUND

Glucose control in diabetics is of paramount importance. While poor glucose control leads to morbidity and associated mortality, good glucose control has been shown to reduce cardiovascular, retinal, and kidney diseases by almost 50%, in addition to considerably reducing other complications [The Diabetes Control and Complications Trial Research Group. N. Engl. J. Med. 329:977-986 (1993)].

The push for better management of glucose control in the past led to the development of conventional hand-held glucose monitors. While the use of such glucose monitors has improved insulin strategy, actual insulin delivery remains inflexible, i.e. a fixed dose via a systematic route. In contrast, the normal physiological insulin delivery system, the pancreatic islet cells, is a much more sophisticated system that allows perfect glucose control by measuring blood glucose and delivering the appropriate insulin into the portal vein on a minute to minute basis.

In order to provide a more flexible and effective means of insulin delivery, insulin pumps were developed in the 1980's. These pumps allowed an individual to dial in a flexible dosage of insulin and led to the development of implantable insulin devices with large insulin reservoirs that need to be replenished only three to four times a year. Such devices are usually placed in the peritoneum to deliver insulin to the portal venous system and are replenished transdermally. Several hundred devices have been implanted into diabetics to date [Olsen, C. L. et al., Diabetes Care 18:70-76 (1995); Buchwald, H. et al., ASAIO J. 40:917-918 (1994); Broussolle, C. et al,. Lancet 343:514-515 (1994); Olsen, C. L. et al., Int. J. Artificial Organs 16:847-854 (1993); Selam, J. L. et al., Diabetes Care 15:877-885 (1992)]. This system of insulin delivery, however, still relies on external monitoring of blood glucose levels and thus has been coined an “open loop system.” The incorporation of an endogenous glucose sensor into this system would render it a “closed loop system” capable of continuous quantitation of glucose and subsequent delivery of an appropriate amount of insulin.

Various methodologies have been employed to create efficient glucose sensors. While glucose sensors have been developed using physical chemical approaches, such sensors tend to lack both specificity and sensitivity. For example, an infrared device has been developed which measures blood glucose, however, this device is reliant on complex computer analysis of the emission spectra to enhance the relatively weak glucose signal and distinguish it from background noise [Robinson, M. R. et al., Clin. Chem. 38:1618-1622 (1992)].

A biological approach to developing glucose sensors offers the advantages of high specificity and sensitivity, and an option of distinguishing different isomers of the same compound. Biological systems are already widely used in clinical chemistry and are also found in all current hand held glucometers, which incorporate the enzyme glucose oxidase into the glucose sensing system. A number of implantable glucose sensor systems have been proposed. For example, U.S. Pat. Nos. 4,650,547; 4,671,288; 4,781,798; 4,703,756; 4,890,620; 5,569,186 and 5,964,993 all disclose implantable enzyme-based glucose sensors. The glucose sensing ability of these implantable devices, like that in conventional hand-held glucometers, is based on the activity of the enzyme glucose oxidase, which catalyses the oxidation of glucose to yield gluconolactone and hydrogen peroxide. The sensors described in the above-listed patents monitor either the consumption of oxygen or the generation of hydrogen peroxide as an indication of glucose concentration.

A major drawback inherent in these systems is the fact that enzyme-catalysed reactions are greatly affected by the concentration, and therefore the availability, of their reactants. Thus, if access of either glucose or oxygen to the device containing the glucose oxidase is compromised in any way, the results obtained from measuring the catalytic activity of the enzyme will be inaccurate. In the blood, for example, the glucose concentration is typically much higher than the concentration of available oxygen, therefore, the rate of the enzyme-catalysed oxidation of glucose will be controlled by the oxygen concentration and will not accurately reflect the concentration of glucose. In addition, since these devices depend upon the enzyme maintaining its catalytic activity, they must be protected from any molecules, such as inhibitors, that may interfere with this enzyme activity. Furthermore, if the device is monitoring hydrogen peroxide generation, it must also be protected from certain endogenous enzymes, such as catalase, that utilise hydrogen peroxide as a substrate.

An implantable glucose oxidase based biosensor has recently been introduced by Medtronic MiniMed in the U.S. Since this sensor also relies on the catalytic activity of the enzyme glucose oxidase, it is subject to the same drawbacks indicated above. This biosensor has been limited to investigational use only by U.S. law.

Other proteins have been proposed as candidate biosensors for glucose. For example, U.S. Pat. No.. 6,197,534 describes engineered proteins for analyte sensing. This patent specifically discloses a glucose/galactose binding protein (GGBP) to which a detectable label has been attached. The detectable quality of the label changes in a concentration-dependent manner upon glucose binding to the protein, thus allowing the presence or concentration of glucose in a sample to be determined. The biosensors described in this patent are proposed for use in hand-held glucometers only.

U.S. Pat. No. 6,277,627 discloses a glucose biosensor comprising a genetically engineered glucose-binding protein (GBP). The GBP is engineered to include mutations that allow the introduction of environmentally sensitive reporter groups, the signal from which changes with the amount of glucose bound to the protein. The biosensors described in this patent are proposed for use in the food industry, in clinical chemistry or as part of an implantable device.

While both U.S. Pat. Nos. 6,19.7,534 and 6,277,627 disclose biosensors to directly measure glucose concentration, which are not reliant upon the catalytic property of an enzyme, they still face certain drawbacks. Of these, the most significant is that both GGBP and GBP, like glucose oxidase, are bacterially derived and are not, therefore, necessarily optimized for detection of physiological concentrations of glucose in a human subject. Both biosensors require incorporation of detectable labels or reporter systems into the protein and the resultant requirement for an appropriate light source for the reporter systems limits the ability of these sensors in an implantable device.

Only a small number of proteins are known that bind glucose. As mentioned above, current protein-based glucose sensors employ bacterially derived proteins, most usually glucose oxidase. Notable drawbacks to the use of this protein include the fact that no known human counterpart exists and thus its use may have unfavourable antigenic consequences. It is also a very large, highly glycosylated protein (186,000 kD), which requires the co-factor flavin mononucleotide for activity. The kinetics of glucose oxidase are unknown and, to date, it has not been cloned.

Known human proteins that bind glucose are either enzymatically active or membrane-bound (i.e. insoluble). Amongst the enzymatically active proteins, glucokinase is an exquisitely specific enzyme that binds only the physiological isomer of glucose (D-glucose), and no other sugars, with real affinity (Km =6 mM). Glucokinase belongs to a family of enzymes known as hexokinases. The structure of human brain hexokinase I has been determined by X-ray crystallography [Aleshin, A. E., et al, Structure, 6:39-50 (1998); Aleshin, A. E., et al, J. Mol. Biol., 282:345-357 (1998)].

Human glucokinase is found in only two tissues, the liver and the β-islet cells of the pancreas, where it is believed to be involved in determining levels of insulin secretion. It is a cytoplasmic protein (i.e. soluble) and both liver and pancreatic isoforms have been cloned [Tanizawa, Y., et al., Mol. Endocrinol., 6:1070-1081 (1992); Koranyi, L. I., et al., Diabetes, 41:807-811 (1992); Tanizawa, Y., et al., Proc. Nat. Acad. Sci. USA, 88:7294-7297 (1991)].

Three isoforms of human glucokinase are known: isoform 1, specific to islet cells, is 465 amino acids in length (GenBank Accession No. P35557), and isoforms 2 and 3, specific to liver cells, include the major form with 466 amino acids (GenBank Accession No. AAB97681) and the minor form with 464 amino acids (GenBank Accession No. AAB97682). The tissue distribution of glucokinase is due to the presence of alternative promoters, which initiate transcription at different loci in the glucokinase gene. These cell-tissue specific promoters dictate very similar cDNAs that differ only at their 5′ ends. Of the 10 exons that make up the cDNA, exons 2-10 are identical in both tissues. However, exon 1 of the transcripts maps to different loci of the glucokinase gene and differs not only in the 5′ untranslated region, but also in the initial 48 nucleotides of the protein coding sequence. Thus the N-terminal ends of the three isoforms of the 52 kD polypeptide differ in their first 14, 15 and 16 amino acids.

Glucokinase catalyses the phosphorylation of glucose to yield glucose-6-phosphate, a reaction that requires ATP as co-substrate. The kinetics of glucokinase activity have been well-studied and demonstrate that binding of glucose to the enzyme occurs independently of ATP binding [Malaisse, W. J., et al., Archives Internationales de Physiologie et de Biochimie, 97:417-425 (1989); Pollard-Knight, D., et al., Biochem. J., 245:625-629 (1987)]. The reaction mechanism is an ordered Bi—Bi sequential mechanism in which the substrate glucose binds first and the product glucose-6-phosphate leaves last.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a glucose sensor comprising a glucokinase protein, wherein the catalytic enzymatic activity has been disabled. The protein retains a high specific affinity for and ability to bind glucose with the appropriate kinetics to be considered as a glucose sensor in a biomedical device.

In accordance with one aspect of the present invention, there is provided a recombinant human glucokinase having decreased catalytic activity but a substantially identical ability to bind glucose relative to the corresponding wild-type human glucokinase.

In accordance with another aspect of the present invention, there is provided an isolated nucleic acid molecule encoding a mutant human glucokinase having decreased catalytic activity but a substantially identical ability to bind glucose relative to the corresponding wild-type human glucokinase.

In accordance with further aspect of the present invention, there are provided vectors comprising an isolated nucleic acid molecule encoding a catalytically disabled human glucokinase and host cells comprising these vectors.

In accordance with another aspect of the invention, there is provided a method of producing a recombinant catalytically disabled human glucokinase comprising culturing a host cell containing a vector encoding the glucokinase under conditions in which the glucokinase is expressed and isolating the expressed glucokinase.

In accordance with another aspect of the invention, there is provided a glucose sensor comprising a recombinant human glucokinase having decreased catalytic activity but a substantially identical ability to bind glucose relative to the corresponding wild-type human glucokinase.

In accordance with a further aspect of the invention, there is provided a method of determining the level of glucose in a sample comprising contacting the sample with a recombinant catalytically disabled glucokinase, measuring a change in a physical characteristic of said recombinant human glucokinase and then correlating this change to the level of glucose in the sample.

### BRIEF DESCRIPTION OF THE FIGURES

FIG. 1 reveals the nucleic acid sequence of the major isoform of human liver glucokinase (SEQ ID NO:1).

FIG. 2 shows the amino acid sequence corresponding to the nucleic acid sequence of FIG. 1 (GenBank Accession No. AAB97681; SEQ ID NO:2).

FIG. 3 shows the amino acid sequence corresponding to the minor isoform of human liver glucokinase (GenBank Accession No. AAB97682; SEQ ID NO:18).

FIG. 4 shows the amino acid sequence corresponding to the pancreatic isoform of human glucokinase (GenBank Accession No. P35557; SEQ ID NO:19).

FIG. 5 is a comparison of the amino acid sequences shown in FIGS. 2, 3 and 4.

FIG. 6 depicts the purification of bacterially-expressed glucokinase fusion proteins. A) Glutathione S-transferase-glucokinase (GST-GLK) fusion proteins were purified from 5 ml cultures of pGEX-GLK-transformed BL21 E. coli using glutathione-agarose beads and subjected to Western blot analysis with an anti-GST antibody. Stable full-length GST-GLK (lane 1) and the GST-mutant glucokinase proteins (Ser336Val, Ser336Leu, Ser336Ile and Asp205Ala; lanes 2-5, respectively) were produced. B) His-tagged glucokinase (His-GLK) was purified from 250 ml cultures of pET-15b-GLK transformed BL21-(DE3)-pLysS E. coli using Ni²⁺-NTA columns (QIAGEN) and subjected to SDS-PAGE and Coomassie Blue staining. Lane 1 contains Rainbow MW markers (0.75 μg protein/band); lane 2, total cell lysate (0.001%); lanes 3 and 4, proteins eluted from the columns with 250 mM imidazole (1%, from duplicate preparations). His-tagged wild-type glucokinase of greater than 95% purity was obtained.

FIG. 7 depicts fluorescence spectroscopy analysis of His-tagged glucokinase. The intrinsic fluorescence intensity spectrum of His-tagged wild-type glucokinase was measured following excitation at 280 nm. The baseline spectrum of His-tagged glucokinase alone is shown (His-GLK, thin dashed line). Addition of 100 mM glucose to the cuvette resulted in an increase in the maximum fluorescence intensity at 312 nm (His-GLK+glc; thick dashed line), which reached a maximum three minutes later (His-GLK+glc reread; thick solid line). The buffer (stippled line) exhibited minimal intrinsic fluorescence.

FIG. 8 depicts fluorescence spectroscopy analysis of His-tagged glucokinase immobilized on Ni-NTA agarose (QIAGEN). The intrinsic fluorescence intensity spectra were measured in the absence and presence of 100 mM glucose following excitation at 280 nm. The baseline spectrum of immobilized His-tagged glucokinase alone is shown (Ni-NTA-His-GLK, thin line). In the presence of glucose, the maximum fluorescence intensity increased (Ni-NTA-His-GLK+glc, thick line). Immobilized His-GLK that had been previously incubated with glucose then washed with glucose-free buffer [(Ni-NTA-His-GLK+glc) washed, thin dashed line)] also showed an upward shift in maximum fluorescence activity in the presence of glucose [(Ni-NTA-His-GLK+glc) washed+glc, thick dashed line)]. The Ni-NTA agarose itself exhibited some intrinsic fluorescence and its presence likely caused broadening of the peaks compared to FIG. 7.

FIG. 9 depicts A) Immobilized-metal affinity chromatography (IMAC). Adjacent histidine residues in His-tagged proteins interact with the Ni²⁺-NTA matrix. B-D) Typical Nyquist diagrams obtained for different electrode surface conditions. The imaginary (Zᵢ) and real (Zᵣ) complex impedence elements were calculated by computer following impedence measurements. B) Modification of screen-printed electrode (SPE) with NTA ligand. Nitrilotriacetic acid (NTA) ligand was coupled to the bare SPE electrode surface using 1-ethyl-3-(3-dimethylaminopropyl)-carbodiimide (EDC) activation. C) Immobilization of glucokinase. The SPE was loaded with Ni²⁺ cations (Ni2+) and His-tagged wild-type glucokinase (GLK) immobilized on the surface. D) Detection of glucose. Upon addition of glucose (Glc 100 mM) to the immobilized His-tagged GLK, a significant shift to the left in the curve was noted.

### DETAILED DESCRIPTION OF THE INVENTION

Definitions

The terminology used herein is for the purpose of describing particular embodiments only and is not intended to limit the scope of the present invention.

Unless defined otherwise or the context clearly dictates otherwise, all technical and scientific terms used herein have the same meaning as commonly understood by one of ordinary skill in the art to which this invention belongs.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

Use of the singular forms “a”, “an”, and “the” include plural references unless the context clearly dictates otherwise. Thus, for example, reference to “a target polynucleotide” includes a plurality of target polynucleotides.

As used in this specification and claims, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation and is synonymous with the term “approximate”.

Terms such as “connected”, “attached” and “linked” may be used interchangeably herein and encompass direct as well as indirect connection, attachment, linkage or conjugation unless the context dictates otherwise.

Where a value is explicitly recited, it is to be understood that values which are about the same quantity or amount as the recited value are also within the scope of the invention, as are ranges based thereon.

As used herein, the terms “molecule”, “compound”, “agent” or “ligand” are used interchangeably and broadly to refer to natural, synthetic or semi-synthetic molecules or compounds. The term “molecule” therefore denotes, for example, chemicals, macromolecules, cell or tissue extracts (from plants or animals) and the like. Non-limiting examples of molecules include nucleic acid molecules, peptides, antibodies, carbohydrates and pharmaceutical agents.

The term “mutation,” as used herein, refers to a deletion, insertion, substitution, inversion, or combination thereof, of one or more nucleotides in a gene.

The term “isolated”, as used herein, refers to a molecule that has been removed from its source or natural environment, such as by a physical or chemical method. Thus, for example, an isolated polynucleotide refers to a polynucleotide that has been extracted or removed from its original material.

The term “purified”, as used herein, refers to a molecule that has been separated from a cellular component. Thus, for example, a “purified protein” has been purified to a level not found in nature. A “substantially pure” molecule is a molecule that is lacking in most other cellular components.

The terms “glucokinase”, glucokinase peptide“, “glucokinase protein” and “glucokinase polypeptide” are used interchangeably in the present application and designate the glucokinase protein or polypeptide in contrast to the nucleic acid coding for this protein or polypeptide.

The term “catalytic activity-disabled” (CAD), as used herein, means that the enzymatic activity of the enzyme (i.e. glucokinase) has been significantly inhibited, such that the glucokinase still binds glucose, but does not catalyze the phosphorylation of glucose to yield glucose-6-phosphate.

The term “CAD-glucokinase,” as used herein, means a glucokinase enzyme in which the catalytic activity has been disabled. In accordance with the present invention, the catalytic activity of the glucokinase enzyme is disabled by genetically engineering one or more appropriate mutations into the enzyme such that the glucokinase still binds glucose, but does not catalyze the phosphorylation of glucose to yield glucose-6-phosphate.

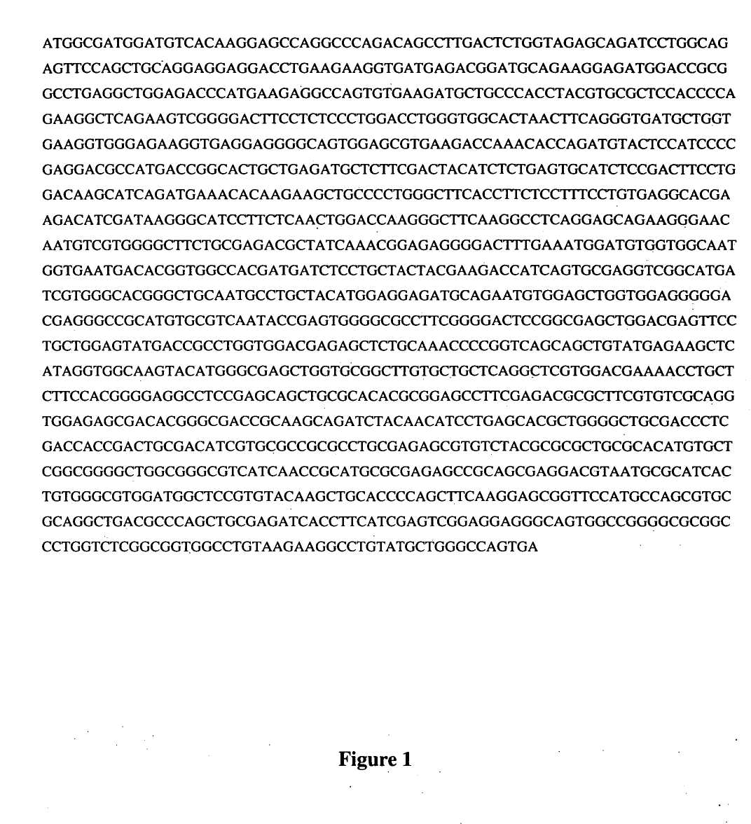

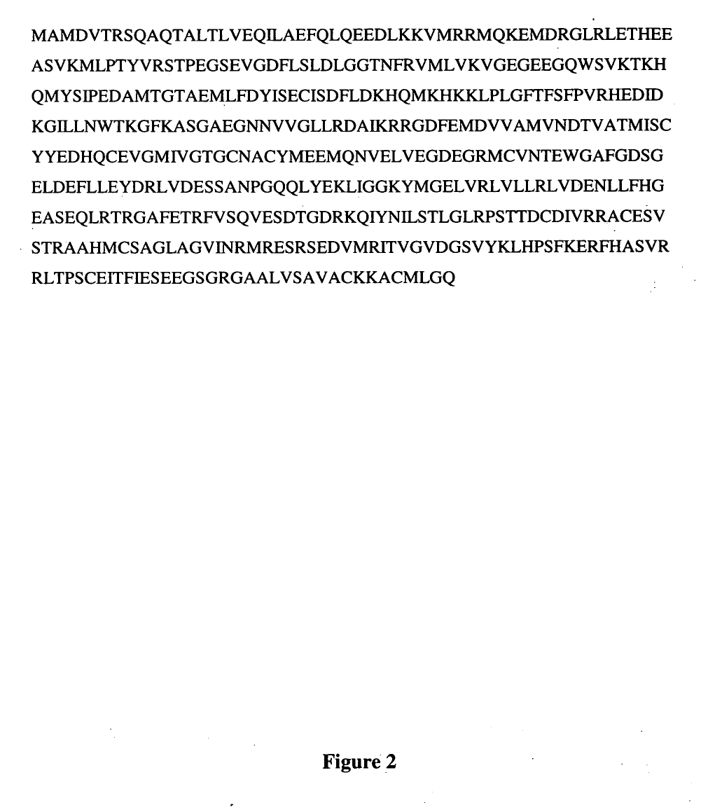

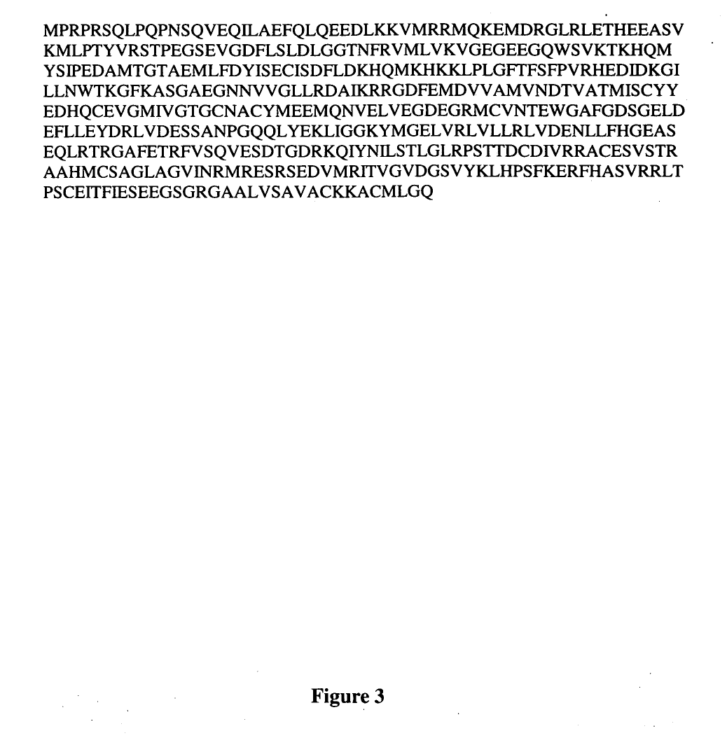

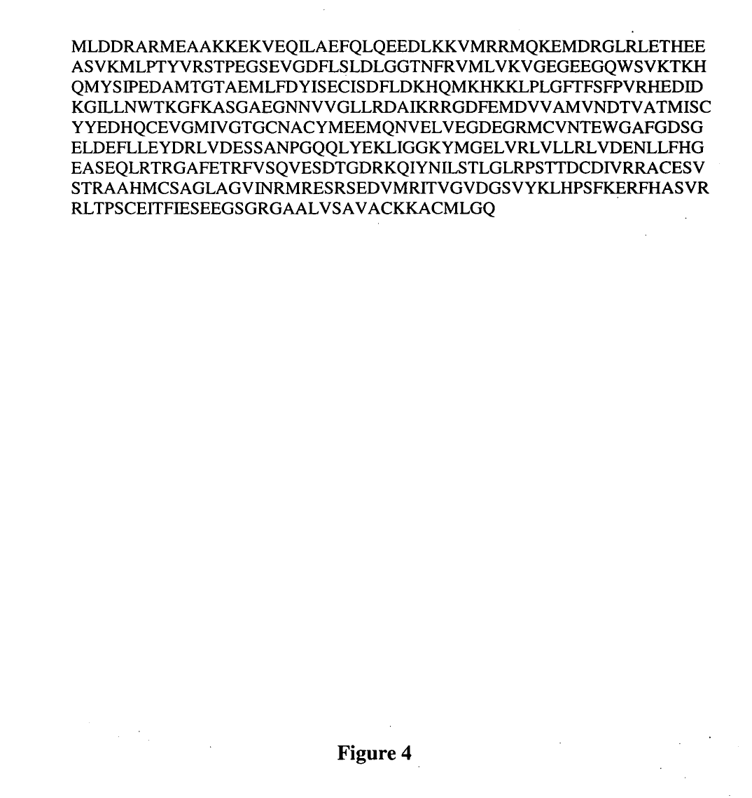

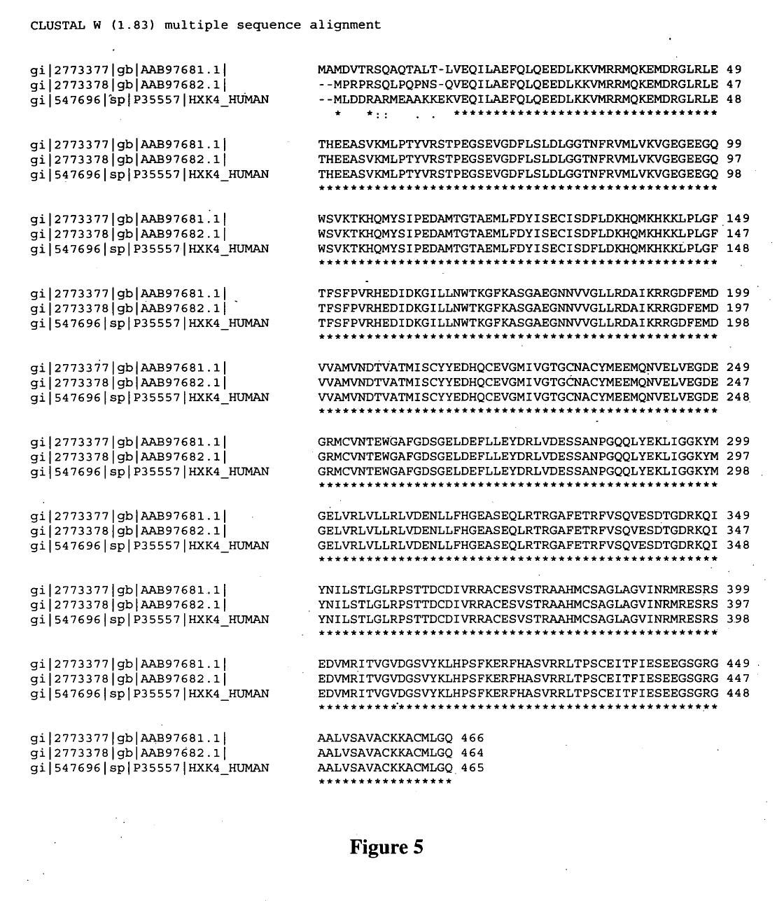

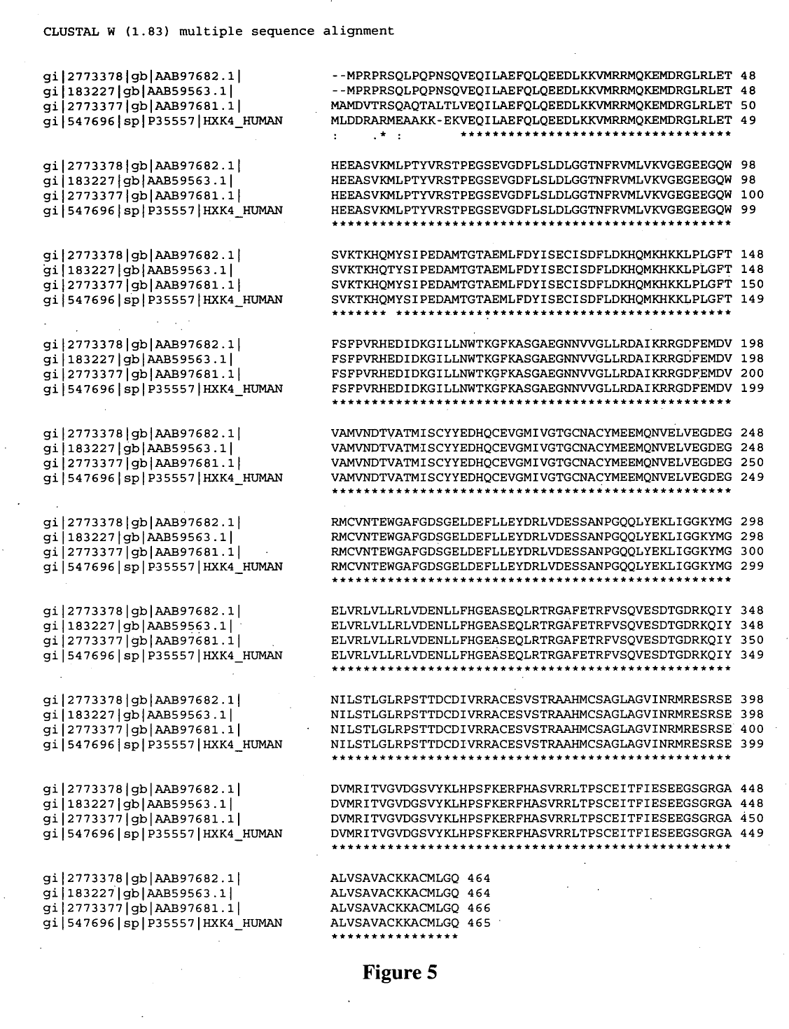

The term “glucokinase”, as used herein, most often refers to the major liver isoform of the enzyme (FIGS. 1 and 2), while the scientific literature mostly refers to the pancreatic isoform (FIG. 4). FIG. 3 shows the amino acid sequence of the minor liver isoform. The major liver isoform of glucokinase differs from the pancreatic form of glucokinase by having one additional amino acid at the amino terminal. Thus, in the present invention, mutations defined by an amino acid found in the pancreatic form in position “X” corresponds with mutations at amino acid “X+1” in the major liver isoform. For example, mutation Asp78 of the pancreatic form is mentioned in the description and the claim language and refers to Asp79 in the major liver isoform. FIG. 5 shows the sequence alignments (or consensus) of the amino acid sequences of the major liver isoform, the minor liver isoform and the pancreatic isoform of glucokinase. Interestingly, there is a second minor liver isoform that is less common. It differs by having a “C” nucleotide instead of a “T” nucleotide in its mRNA, whereas “T” is the more common nucleotide found in humans (see GenBank Accession No M69051). The result is a minor liver glucokinase isoform that differs by one amino acid difference relative to the more common form; see GenBank Accession Nos. AAB97682 and AAB59563.

The term “affinity tag” as used herein refers to a compound with a known affinity for other compounds, where the other compounds are preferably associated with a solid support. Examples of compounds that may be associated with a solid support include include haptens, antibodies, and ligands. A more specific example of a affinity tag is biotin, which can bind to or interact with streptavidin bound to a solid support.

The term “reactive group” as used herein refers to a specific portion of a molecule that is especially sensitive to and chemically reactive with a given site on a different molecule. For example, solid supports may consist of many materials, limited primarily by their capacity to attach to any of a number of chemically reactive groups. Examples of support materials include the type of material commonly used in peptide and polymer synthesis and include glass, latex, polyethylene glycol, heavily cross-linked polystyrene or similar polymers, gold or other colloidal metal particles, and other materials known to those skilled in the art.

Catalytic Activity-Disabled Human Glucokinase Protein

The present invention provides a glucokinase protein in which the enzymatic activity has been disabled in order to enable its use as a glucose sensor. In accordance with the present invention, the enzymatic activity of the glucokinase protein has been significantly inhibited, yet the protein retains a high specific affinity for and the ability to bind glucose. In contrast to known glucose sensors, the catalytic activity-disabled glucokinase (CAD-glucokinase) according to the present invention is derived from a human enzyme and thus is naturally optimised to function throughout the normal physiological range of glucose concentrations. Since the binding of glucose to glucokinase has been shown to occur independently of ATP binding, the catalytic activity-disabled human glucokinase does not require any additional substrates or an energy source in order to bind glucose. In addition, the CAD-glucokinase does not rely on a catalytic reaction to determine glucose concentrations.

Glucose sensors based on the CAD-glucokinase according to the present invention can be used in hand-held monitors, in implantable biosensors or can be incorporated into biomedical devices for continuous glucose monitoring and insulin delivery.

The CAD-glucokinase of the present invention is a recombinant human glucokinase protein that has been genetically engineered to negate the catalytic activity, but to leave the glucose binding properties of the protein largely intact. As the N-terminal differences of the liver and pancreatic isoforms of glucokinase do not have any demonstrable effect on the functional properties of the protein, the present invention contemplates the use of various isoforms of glucokinase for the generation of a CAD-glucokinase.

Thus, in the context of the present.invention, a CAD-glucokinase is provided by introduction of one or more mutations that interfere with the catalytic mechanism of the enzyme and/or interferes with ATP binding. Such effects on the catalytic mechanism or ATP binding can be achieved by deletion and/or substitution of one or more of the amino acids involved, directly or indirectly, in either ATP binding or in catalysis, but not in glucose binding.

As one skilled in the art will appreciate, introduction of a null enzymatic phenotype into the glucokinase creates the potential for ATP binding to the glucokinase to create a ternary complex that may simulate “suicide” or “dead-end” non-competitive inhibition and/or to produce additional conformational changes not related to glucose concentration and/or to interfere with the dissociation of glucose, none of which are desirable in a glucose sensor. The CAD-glucokinase in accordance with one embodiment of the present invention, therefore, is engineered such that the ability to bind ATP is compromised, or abolished. This can be achieved, for example, by mutation of at least one residue involved, directly or indirectly, in ATP binding. Mutation of ATP-binding residues will also help to prevent other related substrates (e.g. inorganic pyrophosphate, PPi) from binding at this site and potentially affecting glucose binding and or causing conformational change.

Many of the catalytically important amino acid residues have been identified in glucokinase, as have many of those involved in both glucose and ATP binding. The residues Lys169, Thr168, Asn231, Asn204, Glu256, and Glu290 have been identified as the main residues constituting the active binding site for glucose in glucokinase [Mahalingam, B., et al., Diabetes, 48:1698-1705 (1999); St. Charles, R, et al., Diabetes, 43:784-791 (1994); Pilkis, S. J., et al., J. Biol. Chem., 269:21925-21928 (1994); Xu, L. Z., et al., J. Biol. Chem., 269:27458-27465 (1994); Lange, A. J., et al., Biochem. J., 277:159-163 (Pt 1) (1991); Takeda, J., et al., J. Biol. Chem., 268:15200-15204 (1993)]. The active amino acids in the ATP-binding cleft include: Gly81, Arg85 and Lys169 (interact with γ-O3 phosphate group); Asp78, Ser151 and Asp205 (interact with Mg²⁺ of Mg-ATP); Thr82, Asn83 and Thr228 (interact with the α-O3 phosphate group); Lys169 (interacts with the β-O3 phosphate group); Ser336 (interacts with the adenine moiety); and Lys296, Thr332 and Ser411 (interact with the ribose moiety). In addition, Asp205 has been identified as the most catalytically important residue, acting as the base catalyst that promotes nucleophilic attack of the 6-hydroxyl group of glucose on the γ-phosphate of ATP. Replacement of this residue with alanine has been shown to result in 1,000-fold reduction of enzyme activity, without a significant change in either glucose or ATP binding affinity [Lange, A. J., et al., Biochemical Journal 277 (Pt 1): 159-63 (1991)].

Furthermore, natural mutations that occur in glucokinase offer a wealth of information regarding structure-function relationships. Missense mutations linked to early onset non-insulin dependent diabetes mellitus (MODY) have been well characterised [Page, R. C., et al., Diabetic Medicine, 12:209-217 (1995); Xu, L. Z., et al., J. Biol. Chem., 270:9939-9946 (1995); Xu, L. Z., et al., J. Biol. Chem., 269:27458-27465 (1994); Shimokawa, K, et al., J. Clin. Endocrinol. Metab., 79:883-886 (1994); Wajngot, A., et al., Diabetes, 43:1402-1406 (1994); Lange, A. J., et al., Biochem. J., 277:159-163 (Pt 1), (1991); Takeda, J., et al., J. Biol. Chem., 268:15200-15204 (1993); Stoffel, M., et al., Proc. Nat. Acad. Sci., USA, 89:7698-7702 (1992)] and support the roles of some of the above-mentioned residues (e.g. the mutations Glu256Lys and Thr228Met both drastically reduce Vₘₐₓ, with Glu256Lys causing a 3-fold decrease in Kₘ for glucose but Thr228Met leaving the Kₘ for glucose unaffected) as well as providing guidance for the selection of appropriate residues to mutate to produce a CAD-glucokinase. Studies of naturally occurring glucokinase mutations in MODY patients have indicated that Val203 and Gly261 residues are important in a glucose-induced fit effect and ATP binding, respectively [Liang, Y, et al., Biochem. J., 309:167-173 (1995)].

Provided with the structure/function information available for glucokinase, one skilled in the art can readily select appropriate amino acids for mutation in engineering a CAD-glucokinase. For example, as indicated above, introduction of a mutation at residue 205 vastly decreases the catalytic efficiency of the enzyme and mutation of one of Asp78, Gly80, Thr209, Gly227, Thr228, Ser336, Gly410, Ser411 or Lys414 has the potential to impact the ATP-binding ability of the glucokinase. Thus, the present invention contemplates genetically engineered glucokinase proteins in which one or more of the above-mentioned residues involved in catalysis or ATP binding, but not in glucose binding, is altered to produce a CAD-glucokinase that retains its ability to bind glucose. The present invention also contemplates the mutation of residues that are not directly involved in catalysis or ATP binding, but which are in close proximity to residues that are and which may thereby indirectly affect catalysis or ATP binding.

As an alternative to rational selection of appropriate residues for mutation, a random approach to generating mutations in the glucokinase can be adopted using techniques known in the art. The resultant mutants can be screened for their ability to bind glucose and the loss of their ability to catalyse the conversion of glucose to glucose-6-phosphate, thereby isolating CAD-glucokinases in accordance with the present invention.

In one embodiment of the present invention, the genetically engineered CAD glucokinase is mutated at residue Asp 205. A specific embodiment of this type of mutation includes Asp 205Ala. In another embodiment, the CAD glucokinase contains a mutation at residue Ser 336. Specific embodiments of this mutation include Ser336Leu, Ser336Val and Ser336Ile.

Means of Disabling the Enzymatic Activity

As is known in the art, genetic engineering of a protein generally requires that the nucleic acid encoding the protein first be isolated and cloned. Sequences for the pancreatic form of human glucokinase are available from GenBank (for example, Accession Nos. AAA52562; AAA51824; NP_{—}000153 [protein] and M90299; M88011; NM_{—}000162 [nucleotide]), as are the sequences for the major liver isoform of human glucokinse (Accession Nos. AAB97681; NP_{—}277042 [protein] and NM_{—}033507 [nucleotide]) and minor liver isoforms of glucokinase (Accession Nos. AAB97682; NP_{—}277043; AAB59563 [protein] and NM_{—}033508; M69051 [nucleotide]). Isolation and cloning of the nucleic acid sequence encoding the human glucokinase can thus be achieved using standard techniques [see, for example, Ausubel et al., Current Protocols in Molecular Biology, Wiley & Sons, NY (1997 and updates); Sambrook et al., Molecular Cloning: A Laboratory Manual, Cold-Spring Harbor Press, NY (2001)]. For example, the nucleic acid sequence can be obtained directly from a suitable human tissue, such as liver or pancreatic tissue or an insulinoma, by extracting the mRNA by standard techniques and then synthesizing cDNA from the mRNA template (for example, by RT-PCR). Alternatively, the nucleic acid sequence encoding human glucokinase can be obtained from an appropriate human cDNA library by standard procedures. The isolated cDNA is then inserted into a suitable vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The vector may be a cloning vector or it may be an expression vector. Procedures for cloning human glucokinase are also described in the literature [Koranyi, L. I., et al., Diabetes, 41:807-811 (1992); Tanizawa, Y., et al., Proc. Nat. Acad. Sci., USA, 88:7294-7297 (1991)]. Alternatively, the cloned human pancreatic glucokinase coding sequence can be obtained from the American Type Culture Collection (ATCC) (see ATCC No. 79040 or 79041), as can the cloned glucokinase coding sequence isolated from liver carcinoma (see ATCC No. MGC-1742).

The present invention contemplates the use of one of the known isoforms of glucokinase in the creation of a genetically engineered, CAD-glucokinase as well as those isoforms that may be identified in the future. As mentioned previously, the difference between the cDNA of the liver and the pancreatic isoforms of glucokinase is only at the 5′ end of the cDNA. Therefore, one skilled in the art will appreciate that, once the cDNA of one isoform has been cloned, other isoforms can be readily engineered by addition and/or deletion of the appropriate nucleotides using standard molecular biological techniques.

In one embodiment of the present invention, the CAD-glucokinase is produced from one of the human liver glucokinase isoforms. In another embodiment, the CAD-glucokinase is produced from human liver glucokinase isoform 2. In another embodiment, the CAD-glucokinase is produced from the human pancreatic glucokinase isoform.

Once the nucleic acid sequence encoding human glucokinase has been obtained, mutations can be introduced at specific, pre-selected locations by in vitro site-directed mutagenesis techniques well-known in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

If desired, after introduction of the appropriate mutation or mutations, the nucleic acid sequence encoding human glucokinase can be inserted into a suitable expression vector. Examples of suitable expression vectors include, but are not limited to, plasmids, phagemids, cosmids, bacteriophages, baculoviruses and retroviruses, and DNA viruses. In one embodiment of the present invention, the nucleic acid encoding the genetically engineered glucokinase is cloned into a baculovirus plasmid.

One skilled in the art will understand that the expression vector may further include regulatory elements, such as transcriptional elements, required for efficient transcription of the glucokinase coding sequences. Examples of regulatory elements that can be incorporated into the vector include, but are not limited to, promoters, enhancers, terminators, and polyadenylation signals. The present invention, therefore, provides vectors comprising a regulatory element operatively linked to a nucleic acid sequence encoding a genetically engineered, CAD-glucokinase. One skilled in the art will appreciate that selection of suitable regulatory elements is dependent on the host cell chosen for expression of the genetically engineered glucokinase and that. such regulatory elements may be derived from a variety of sources, including bacterial, fungal, viral, mammalian or insect genes.

In the context of the present invention, the expression vector may additionally contain heterologous nucleic acid sequences that facilitate the purification of the expressed glucokinase. Examples of such heterologous nucleic acid sequences include, but are not limited to, affinity tags such as metal-affinity tags, histidine tags, biotin tags, avidin/strepavidin-encoding sequences and glutathione-S-transferase (GST) encoding sequences.

The expression vectors can be introduced into a suitable host cell or tissue by one of a variety of methods known in the art. Such methods can be found generally described in Ausubel et al., Current Protocols in Molecular Biology, Wiley & Sons, NY (1997 and updates); Sambrook et al., Molecular Cloning: A Laboratory Manual, Cold-Spring Harbor Press, NY (2001) and include, for example, stable or transient transfection, lipofection, electroporation, and infection with recombinant viral vectors. One skilled in the art will understand that selection of the appropriate host cell for expression of the genetically engineered glucokinase will be dependent upon the vector chosen. Examples of host cells include, but are not limited to, bacterial, yeast, insect, plant and mammalian cells.

Methods of cloning and expressing proteins are well-known in the art, detailed descriptions of techniques and systems for the expression of recombinant proteins can be found, for example, in Current Protocols in Protein Science (Coligan, J. E., et al., Wiley & Sons, New York).

The CAD-glucokinase can be purified from the host cells by standard techniques known in the art. If desired, the changes in amino acid sequence engineered into the protein can be determined by standard peptide sequencing techniques using either the intact protein or proteolytic fragments thereof.

As an alternative to a directed approach to introducing mutations into glucokinase, a cloned glucokinase gene can be subjected to random mutagenesis by techniques known in the art. Subsequent expression and screening of the mutant forms of the enzyme thus generated would allow the identification and isolation of CAD-glucokinases.

The present invention also contemplates fragments of the CAD-glucokinase, for example, fragments that comprise the glucose binding domain, which retain the ability to bind glucose but do not catalyse its conversion to glucose-6-phosphate. Such fragments can be readily generated for example, by cloning a fragment of the gene encoding the full-length CAD-glucokinase. Fusion proteins comprising a fragment of a CAD-glucokinase and a heterologous amino acid sequence are also contemplated. Examples of such heterologous amino acid sequences include those encoding an affinity tag, epitope, marker, reporter protein, or the like.

The present invention, therefore, provides isolated nucleic acid molecules encoding a CAD-glucokinase, or a fragment or domain thereof, vectors comprising such nucleic acids as well as host cells comprising the vectors.

Functional Criteria of the Catalytic Activity-Disabled Glucokinase

In the context of the present invention, to be useful as a glucose sensor the catalytic activity of the glucokinase is disabled (i.e. the protein does not exhibit significant catalytic activity with respect to the conversion of glucose to glucose-6-phosphate), yet the glucokinase retains the ability to specifically bind glucose with an affinity approaching that of the wild-type enzyme and optionally has significantly reduced or abolished ability to bind ATP.

I. Catalytic Activity

The catalytic activity of the CAD-glucokinase is determined by measuring the ability of the protein to catalyze the phosphorylation of glucose in the presence of ATP. The extent to which the catalytic activity of the CAD-glucokinase has been impaired is then determined by comparison of the measured activity to that of the wild-type enzyme.

Methods of assaying the catalytic activity of hexokinases are known in the art. Assays to measure the activity of glucokinase can be generally based on that described by Storer [Storer, A. C., et al., Biochem. J., 141:205-209 (1974)] which utilises a coupled enzymatic assay employing glucose-6-phosphate dehydrogenase leading to the production of NADPH. The amount of NADPH produced in the assay can readily be measured by monitoring the increase in absorbance at 340 nm. One skilled in the art will appreciate that modifications can be made to the basic assay if desired [for example, see Trifiro, M., et al., Prep. Biochem 16:155-173 (1986)].

In general, preparations of the wild-type or CAD-glucokinase are added to a buffered reaction mixture containing NADP, potassium chloride, glucose-6-phosphate dehydrogenase, glucose and ATP. Phosphorylation of the glucose to glucose-6-phosphate by the glucokinase and subsequent reduction of glucose-6-phosphate and production of NADPH by the glucose-6-phosphate dehydrogenase leads to an increase in absorbance at 340 nm, which is monitored as an indication of the amount of NADPH produced. This value can then be correlated to the activity of the glucokinase or CAD-glucokinase by standard methods.

Glucokinase activity is generally defined in units per millilitre, where one unit of activity is the amount of enzyme that transforms, under optimal conditions, 1 μmole of substrate/min at room temperature. In the context of the present invention a CAD-glucokinase protein is one that has an activity that is between 10 and 10 000-fold less than that of the wild-type enzyme. In one embodiment of the present invention, the activity of the CAD-glucokinase is decreased by between 100 and 10 000-fold when compared to the wild-type enzyme. In another embodiment, the activity of the CAD-glucokinase is decreased by at least 1 000-fold when compared to the activity of the wild-type enzyme.

II. Binding Affinity for Glucose and ATP

The ability of the CAD-glucokinase to bind glucose with an affinity approaching that of the wild-type enzyme is essential. A CAD-glucokinase with an impaired ability to bind glucose will be unable to function efficiently as a glucose sensor.

The binding affinity of the CAD-glucokinase for glucose and ATP can be determined by techniques well-known in the art. The measured binding affinities can then be compared to those of the wild-type enzyme to provide an indication of the extent to which the binding affinities have been affected. Methods of measuring binding affinities are known in the art [for example, see Liang, Y., et al., Biochem. J., 309:167-173(1995); Shkolny, D. L., et al., J. Clin. Endocrinol. Metab., 84:805-810 (1999)]. In general, the appropriate substrate (i.e. glucose or ATP) is first labelled with a detectable label. The wild-type glucokinase or CAD-glucokinase is then mixed with various concentrations of the labelled substrate and the amount of bound substrate is determined. Results are analysed by standard methods, for example through the use of Scatchard plots, and the binding affinities of the wild-type enzyme and the CAD-glucokinase are compared.

Detectable labels are moieties a property or characteristic of which can be detected directly or indirectly. One skilled in the art will appreciate that the detectable label is chosen such that it does not affect the ability of the wild-type protein to bind the substrate. Labels suitable for use with the substrates include, but are not limited to, radioisotopes, fluorophores, chemiluminophores, colloidal particles, fluorescent microparticles and the like. Examples of suitable labelled substrates include, but are not limited to, trinitrophenyl (TNP)-ATP (Molecular Probes, Eugene, Oreg.), D-glucose 2-3H (NEN, Boston, Mass.) and ³²P α-ATP (NEN, Boston, Mass.). One skilled in the art will understand that these labels may require additional components, such as triggering reagents, light, and the like to enable detection of the label. In one embodiment of the present invention, the substrates are labelled with a radioisotope. In another embodiment, the substrates are labelled with the radioisotope ³H.

In accordance with the present invention, the CAD-glucokinase retains at least 10% of the binding affinity for glucose that is measured for the wild-type enzyme. In one embodiment, the CAD-glucokinase retains at least 20% of the wild-type binding affinity for glucose. In another embodiment, the CAD-glucokinase retains at least 30% of the wild-type binding affinity for glucose. In other embodiments, the CAD-glucokinase retains at least 40% and at least 50% of the wild-type binding affinity for glucose.

In one embodiment of the present invention, the ability of the CAD-glucokinase to bind ATP is either abolished or impaired. Since it has been demonstrated that ATP binding is not required in order for glucokinase to bind glucose, disabling the ATP-binding ability of the protein by site-directed mutagenesis will prevent the enzyme from completing the phosphorylation reaction and will thus contribute to its lack of enzymatic activity, but will not interfere with the glucose-binding ability of the protein. In addition, removal of the ATP-binding ability will help to prevent the formation of any dead-end ternary complexes by the protein.

In accordance with one embodiment of the present invention, therefore, the CAD-glucokinase has less than 50% of the binding affinity for ATP that is measured for the wild-type enzyme. In one embodiment, the CAD-glucokinase has less than 40% of the wild-type binding affinity for ATP. In other embodiments, the CAD-glucokinase retains less than 30%, less than 20% and less than 10% of the wild-type binding affinity for ATP.

III. Dissociation Parameters

The ability of the CAD-glucokinase to release glucose or allow glucose to dissociate in a specific time frame is an important issue. If the CAD-glucokinase forms long-lasting glucose-glucokinase complexes, then its ability to sense changing glucose concentrations in relatively short time frames will be jeopardized.

Measurement of parameters such as the dissociation rate (k) for glucose or the half-lives (t_{1/2}, i.e. the time required for 50% of bound glucose to dissociate) of glucose-glucokinase complexes provides an indication of the ability of the CAD-glucokinase to release glucose. Comparison of the value of these parameters with those for the wild-type glucokinase indicates whether this ability is impaired. Determination of the above parameters can be readily achieved by a worker skilled in the art using standard techniques [for example, see Shkolny, D. L., et al., J. Clin. Endocrinol. Metab., 84:805-810 (1999)].

For example, the dissociation rate of a substrate or ligand can be measured by standard dissociation binding experiments using a labelled substrate/ligand. In general, the protein and the labelled substrate are allowed to bind, usually to equilibrium, and then further binding of the labelled substrate is blocked. The rate of dissociation of the labelled substrate from the protein is measured by determining how much substrate remains bound at various time points subsequent to the blocking step. Further binding of the labelled substrate can be blocked by a number of methods, for example, the protein can be attached to a suitable surface and the buffer containing the labelled substrate can be removed and replaced with fresh buffer without labelled substrate. Alternatively, a very high concentration of unlabelled substrate can be added, the high concentration of unlabelled substrate ensures that it instantly binds to nearly all the unbound protein molecules and thus blocks binding of the labelled substrate, or the suspension can be diluted by a large factor, for example 20- to 100-fold, to greatly reduce the concentration of labelled substrate such that any new binding of labelled substrate by the protein will be negligible.

In one embodiment of the present invention, the dissociation constants are determined using glucose radiolabelled with 3H as the substrate and addition of non-radioactive glucose is used to block further binding of the radiolabelled glucose. At various times, aliquots are removed and the amount of bound and free ³H-glucose is determined.

Rates of dissociation are generally expressed as the fraction of complexes dissociating per unit time and as half-lives of complexes. In accordance with the present invention, the dissociation rate for the CAD-glucokinase is in the order of minutes. In one embodiment, the dissociation rate is 0.1 to 10 minutes (e.g. k=0.1/min to k=0.9/min).

One skilled in the art will appreciate that dissociation kinetics can also be measured in real time using surface plasmon resonance (for example, using BIACORE® technology; Biacore International AB, Uppsala, Sweden). As is known in the art, surface plasmon resonance (SPR) occurs when surface plasmon waves are excited at a metal/liquid interface and enables the monitoring of binding events between two or more molecules in real time. Light is directed at, and reflected from, the side of a surface that is not in contact with a sample and, at a specific combination of wavelength and angle, SPR causes a reduction in the reflected light intensity. Biomolecular binding events cause changes in the refractive index at the surface layer, which are detected as changes in the SPR signal. Advantages to measuring real-time dissociation kinetics include the ability to confirm classical dissociation kinetics and as well as providing real-time kinetic information that is important in establishing the suitability of a CAD-glucokinase as a potential glucosensor [see, Malmqvist, M., Biochem. Soc. Trans., 27:335-339 (1999)].

Use of the Catalytic Activity-Disabled Glucokinase as a Glucose Sensor

In accordance with the present invention, the CAD-glucokinase can be used as a glucose sensor, for example, in a hand-held or an implantable glucose-sensing device. The CAD-glucokinase is also suitable for use as the glucose sensor in biomedical devices designed to continuously monitor blood glucose levels and administer insulin.

To function effectively as a glucose sensor, the CAD-glucokinase according to the present invention must possess a measurable characteristic which allows free protein to be distinguished from the glucose-bound protein. Associated with this characteristic, there must additionally be a detectable quality that changes in a concentration-dependent manner when the protein is bound to glucose. An example of one such characteristic is the conformational change that occurs when glucokinase binds glucose.

Conformational Analysis of the CAD-Glucokinase

In one embodiment, the present invention takes advantage of the change in conformation which occurs when glucose binds to glucokinase [Gidh-Jain, M., et al., Proc. Natl. Acad. Sci., USA, 90:1932-1936 (1993); Lin, S. X., et al., J. Biol. Chem., 265:9670-9675 (1990); Neet, K. E., et al., Biochemistry, 29:770-777 (1990); Steitz, T. A., et al., Phil. Trans. Royal Soc. London—Series B: Biological Sciences, 293:43-52 (1981); Pickover, C. A., et al., J. Biol. Chem., 254:11323-11329 (1979); McDonald, R. C., et al., Biochemistry, 18:338-342 (1979); Olvarria, J. M., et al., Archivos de Biologia y Medicina Experimentales, 18: 85-292 (1985); Xu, L. Z., et al., Biochemistry, 34:6083-6092 (1995)]. Such a change in conformation is measurable and thus provides a characteristic that will allow free glucokinase and glucose-glucokinase complexes to be distinguished. Conformational changes of proteins have been demonstrated as a basis for biosensing [Wilner B., Nature Biotech., 19:1023-1024 (2001); Benson D. E., et al., Science, 293:1641-1644 (2001)].

The ability of the CAD-glucokinase to undergo a similar conformational change to the wild-type enzyme upon glucose binding can be confirmed by a number of techniques known in the art. For example, partial proteolytic digestion can be used to indicate the folded state of a protein. As is known in the art, any given protease exhibits a certain bond specificity and thus, when used to digest an unfolded protein, will yield a defined set of peptide fragments which can be separated and analyzed, for example by denaturing polyacrylamide gel electrophoresis (PAGE). However, when the treated protein is in a folded or native state, many of the susceptible bonds may be buried within the hydrophobic core of the protein and thus be inaccessible to the protease. The conformational state of the protein, therefore, defines which bonds will be cleaved and consequently, the pattern of peptide fragments produced. Areas most likely to contain susceptible bonds are exposed loops within domains or the linking regions between domains. These accessible regions could be constantly present, or could arise transiently as a result of the protein undergoing a conformational change.

Partial proteolytic digestion has been used to document successfully several protein conformational states and/or changes in conformation [Inoue, S., et al., J. Biochem., 118:650-657 (1995); Hockerman, G. H., et al., Mol. Pharmacol., 49:1021-1032 (1996); Chen, G. C., et al., J. Biol. Chem., 269:29121-29128 (1994)]. More recently, partial proteolytic digestion has been used to document ligand-induced conformation change of several steroid receptors [Couette, B., et al., Biochem. J., 315:421-427 (1996); Kuil, C. W., et al., J. Biol. Chem., 270:27569-27576 (1995); Kuil, C. W., Mulder, E., Mol. Cell. Endocrinol., 102:R1-R5 (1994); Keidel, S., et al., Mol. Cell. Biol., 14:287-298 (1994); Leng, X., et al., J. Steroid Biochem. Mol. Biol., 46:643-661 (1993); Allan, G. F., et al., J. Biol. Chem., 267:19513-19520 (1992); Kallio, P. J., et al., Endocrinology, 134:998-1001 (1994)].

Partial protease digestion and analysis of resultant peptide fragments, therefore, can be used to demonstrate the conformational change of wild-type glucokinase induced by glucose binding. Once the peptide fragment patterns have been determined for the wild-type glucokinase with and without bound glucose, the peptide fragments generated by partial proteolytic digestion of a CAD-glucokinase protein can then be analysed to determine whether these proteins undergo a similar conformational change. CAD-glucokinase proteins that mimic the conformational changes seen in the wild-type glucokinase can thereby be selected.

Alternatively, a similar technique known as zero-order cross-linking can be used. This technique relies on the activity of the enzyme transglutaminase to cross-link lysine and glutamine residues in the protein that are close together in three-dimensional space. Lysine and glutamine residues that are spatially separated will not be affected by the activity of this enzyme. Pre-treatment of a protein with transglutaminase followed by complete digestion with a protease, such as trypsin, thus yields a “fingerprint” of peptide fragments that can be resolved by standard techniques such as denaturing polyacrylamide gel electrophoresis (see, for example, Safer, D., et al., Biochemistry, 36:5806-5816 (1997)]. Zero-order cross-linking, therefore, can be used to determine the digestion pattern of wild-type glucokinase with or without bound glucose. The pattern of peptides produced from digestion of the CAD-glucokinase proteins pre-treated with transglutaminase can be compared to those of the wild-type protein and those proteins displaying proteolytic peptide fragment patterns similar to those of the wild-type protein can be selected.

A further method that can be used to determine conformational change in the wild-type and catalytic activity-disabled proteins makes use of the redistribution of surface electrical charges that result from large conformational changes in proteins. As is known in the art, most proteins possess a net electrical charge or dipole. Movement of the protein, for example, as the result of binding a substrate, inhibitor or activator, can lead to a change in the overall dipole of the protein, which can be reflected by measurement of simple electrical parameters [see, for example, Mi, L. Z., et al., Biophys. J., 73:446-451 (1997)]. Dielectric relaxation'spectroscopy is a standard method of determining dielectric properties of proteins [see, Biophysical Chemistry, Chapter 14E and F, ed. Marshall Allan G, John Wiley & Sons, Inc. NY. (1978)]. In one embodiment of the present invention, dielectric relaxation spectroscopy employing frequency domain or time domain methodology, such as that described by Smith [Smith, G., et al., J. Pharm. Sci., 84:1029 1044 (1995)], is used to determine the dielectric properties and, therefore, the dipole of the wild-type and CAD-glucokinase.

In addition, the use of newer methods such as NMR and X-ray databases [see, for example, Takashima, S., Biopolymers, 54:398-409 (2001)] to determine the dipole of the wild-type and CAD-glucokinase is also contemplated by the present invention.

Alternatively, the conformational change induced by glucose binding to the wild-type and CAD-glucokinase proteins could be compared using BIACORE® technology (Biacore International AB, Uppsala, Sweden), which uses surface plasmon resonance (SPR) as described previously with respect to the measurement of binding affinities for the CAD-glucokinase.

In order to determine conformational changes in the glucokinase protein upon glucose binding using BIACORE® technology, the protein is first immobilized on a sensor surface. This sensor surface forms one wall of a flow cell and a solution containing glucose is injected over this surface in a precisely controlled flow. Fixed wavelength light is directed at the sensor surface and binding events are detected as changes in the particular angle where SPR creates extinction of light. This change is measured continuously and recorded as a sensorgram. After injection of the glucose-containing solution, a continuous flow of buffer is passed over the surface and the dissociation of the glucose from the glucokinase molecule can be determined. The present invention therefore contemplates the use of BIACORE® technology to determine conformational changes in the catalytic activity-disabled proteins, as well as their binding affinity for glucose and dissociation parameters.

BIACORE® technology is known in the art, as are methods of immobilizing proteins on inert surfaces. Appropriate sensor chips for use in these techniques are commercially available from Biacore International AB (Uppsala, Sweden).

Conformational changes can also be determined in proteins through the use of reporter groups. In one embodiment of the present invention, one or more reporter groups are associated with the CAD-glucokinase. The reporter group can be covalently or non-covalently associated with the protein. Glucokinase proteins that have been further genetically engineered to allow incorporation of a reporter group, for example by inclusion of one or more cysteine residues to provide reactive thiol groups are, therefore, also considered to be within the scope of the present invention. In accordance with the present invention, the reporter group is incorporated into the protein such that it produces a detectable signal when the protein undergoes a conformational change.

One skilled in the art will understand that a variety of reporter groups are available and are suitable for use in the present invention. These reporter groups differ in the physical nature of signal transduction (e.g., fluorescence, electrochemical, nuclear magnetic resonance (NMR), or electron paramagnetic resonance (EPR)) and in the chemical nature of the reporter group. Examples of suitable reporter groups include, but are not limited to, fluorescent reporter groups, non-fluorescent energy transfer acceptors, and the like. Alternatively, the reporter may comprise an energy donor moiety and an energy acceptor moiety, each bound to the glucokinase protein and spaced such that there is a change in the detectable signal when the glucokinase is bound to glucose.

When the glucose sensor comprising the CAD-glucokinase is to be incorporated into an implantable device, fluorophores that operate at long excitation and emission wavelengths (e.g., >600 nm) are most useful (human skin being opaque below 600 nm). Presently, there are only a few environmentally sensitive probes available in this region of the spectrum, although others are likely to be developed in the future that are also suitable for use in the present invention. Examples of those available include, thiol-reactive derivatives of osmium (II) bisbipyridyl complexes and of the dye Nile Blue [Geren, L., et al., Biochem., 30:9450-9457 (1991)]. Osmium (II) bisbipyridyl complexes have absorbances at wavelengths longer than 600 nm with emission maxima in the 700 to 800 nm region [Demas, J. N. et al., Anal. Chem., 63:829-837 (1991)] and long life-times (in the 100 nsec range), simplifying the fluorescence life-time instrumentation. The present invention further contemplates the use of redox cofactors as reporter groups, e.g., ferrocene and thiol-reactive derivatives thereof. Thiol-reactive derivatives of organic free radicals such as 2,2,6,6-tetramethyl-1-piperinoxidy (TEMPO) and 2,2,5,5-tetramethyl-1-piperidinyloxy (PROXYL) can also be used and changes in the EPR spectra of these probes in response to ligand binding can be monitored.

Incorporation of the Catalytic Activity-Disabled Glucokinase within a Biosensor

The technology described here is based on the ability of a biosensor to distinguish unoccupied glucokinase from glucose-bound glucokinase. The approach takes advantage of large changes in glucokinase conformation seen when glucose binds glucokinase [Lin S. X., Neet K. E., J. Biol. Chem. 265:9670-9675 (1990); Neet K. E., et al., Biochemistry 29:770-777 (1990); Steitz T. A., et al., Philos Trans R Soc Lond B Biol Sci 293:43-52 (1981); Pickover C. A., et al., J. Biol. Chem. 254:11323-11329 (1979); McDonald R. C., et al., Biochemistry 18:338-342 (1979); Xu L. Z., et al., Biochemistry 34:6083-6092 (1995); and Olvarria J. M., et al., Archivos de Biologia y Medicina Experimentales 18:285-292 (1985)]. This has been documented using fluorescence spectroscopy experiments [Lin S. X., Neet K. E., J. Biol. Chem. 265:9670-9675 (1990); and Xu L. Z., et al., Biochemistry 34:6083-6092 (1995)].

Most proteins possess a net electrical charge or dipole. Conformational movement of the protein, for example, as the result of binding a substrate, can lead to a change in the overall dipole of the protein [Mi L. Z., et al., Biophys. J. 73:446-451 (1997); Takashima S., Biopolymers 58:398-409 (2001); and Berggren C., et al., Electroanalysis 13:173-180 (2001)], which can be measured by impedance biosensors: impedance changes produced by binding of target molecules to receptor molecules immobilised on the surface of microelectrodes [Katz E., Willner I., Electroanalysis 15:913-947 (2003); Jiang D., et al., Biosens. Bioelectron. 18:1183-1191 (2003); Long Y., et al., J. Colloid. Interface Sci. 263:106-112 (2003); Long Y., et al., J. Biotechnol. 105:105-116 (2003); and Cloarec J. P., et al., Biosens. Bioelectron. 17:405-412 (2002)]. The allosterically-controlled electrochemical transduction of a conformational change induced by a protein-ligand interaction represents a major advance in the development of reagentless biosensors.

In the context of the present invention, a microelectrode consists of a multilayer substrate comprising a conductive base layer and an optional self-assembled monolayer (or other chemical entity) directly or indirectly bound to the conductive base layer. Various conducting or semiconducting substances are known in the art and are suitable for use as the conductive base layer of the microelectrode. Examples include, but are not limited to, gold, silver, and copper (which bind thiol, sulphide or disulphide functional compounds), silicon (either SiH surface which binds alcohols and carboxylic acids, or SiO₂ surface which binds silicon-based compounds such as trichlorosilanes), aluminium, platinum, iridium, palladium, rhodium, mercury, osmium, ruthenium, gallium arsenide, indium phosphide, and mercury cadmium telluride. Examples of suitable forms include foils (such as aluminium foil), wires, wafers (such as doped silicon wafers), chips, semiconductor devices and coatings (such as silver and gold coatings) deposited by known deposition processes.

Self-assembled monolayers (SAMs) are also known in the art and are generally defined as a type of molecule that can bind or interact spontaneously or otherwise with a metal, metal oxide, glass, quartz or modified polymer surface in order to form a chemisorbed monolayer. A self-assembled monolayer should be the thickness of a single molecule (i.e., it is ideally no thicker than the length of the longest molecule included therein). Each of the molecules making up a self-assembled monolayer thus includes a reactive group that adheres to the conductive base layer and may also include a second reactive moiety that can be used to immobilize the protein onto the microelectrode. The microelectrode can alternatively be constructed without the use of SAMs (i.e., by direct physical absorption of the protein onto the conductive layer).

The present invention, therefore, contemplates the immobilization of the CAD-glucokinase onto a microelectrode for use as an impedance biosensor. Methods of immobilizing proteins are well-known in the art (for general techniques, see for example, Coligan et al., Current Protocols in Protein Science, Wiley & Sons, NY). Such immobilization generally makes use of reactive groups on the surface to which the protein is to be attached and/or coupling reagents, such as carbodiimide, succinimides, thionyl chloride, p-nitrophenol, glutaraldehyde, cyanuric chloride and phenyl diisocyanate. One skilled in the art will understand that when a coupling reagent is used, its selection is dependent on the chemical nature of the group on the surface to which the protein is to be immobilized.

The present invention also contemplates the use of CAD-glucokinase proteins which have been further engineered to incorporate an affinity tag or reactive group that facilitates immobilization of the protein to a solid surface. Examples of such affinity tags or reactive groups include, but are not limited to, hexa-histidine tags allowing immobilization onto Ni²⁺-containing surfaces, arsenic or other metal-binding motifs to allow immobilization onto a surface containing the cognate metal, glutathione-S-transferase fusions that allow immobilisation onto glutathione-containing surfaces, avidin or biotin tags and the like. Thus, CAD-glucokinase proteins engineered to incorporate a affinity tag or reactive group that facilitates immobilization of the protein are considered to be within the scope of the present invention. One skilled in the art will appreciate that such an affinity tag or reactive group should not interfere with the binding of glucose by the CAD-glucokinase.

Various biosensors suitable for impedimetric-based sensing have been described in the art. For example, an immunobiosensor has been developed to measure staphylococcus enterotoxin B [DeSilva, M. S., et al., Biosensors & Bioelectronics, 10:675-682 (1995)]. This biosensor contains staphylococcus enterotoxin B antibodies immobilized on an ultra thin platinum film sputtered onto a 100 μm thick silicon dioxide layer within a silicon chip. The film can be considered to be a collection of tiny capacitors connected in series and parallel over the film area. The impedance of this film is extremely sensitive to small changes in the electrical properties of the material between the enterotoxin B antibodies. Binding of enterotoxin B to enterotoxin B antibodies redistributes significant charges on the surface of the antibodies, which in turn decreases the observed impedance.

Similarly, U.S. Pat. No. 5,567,301 describes an immunobiosensor comprising an antibody covalently bound to a substrate material and a pair of electrodes. The biosensor is made by covalently binding the desired antibodies to an ultra-thin metal film sputtered onto a silicon chip. Further examples include the use of proteins immobilized on monomolecular alkylthiol films on gold electrodes [Mirsky et al., Biosens. Bioelectron. 12:977-989 (1997)]; a microfabricated biosensor chip that includes integrated detection elements and within which antibodies are attached to a capture surface (U.S. Patent Application No. 20010053535); and a sensor which uses an affinity component capable of interacting with analyte species and which is immobilized onto a conducting polymer such that the interaction between the affinity component and the analyte induces change in the electrical properties of the polymer (U.S. Pat. No. 6,300,123). Bioaffinity devices have also been described that are based on dipole moment changes [for example, see Hianik, T., et al., Biochem. Bioenerg., 47:47-55 (1998); Mulloni, V., et al., Physica Status Solidi, 182:479-484 (2000); DeSilva, M. S., et al., Biosensors & Bioelectronics, 10:675-682 (1995)].

The present invention, therefore, provides a biosensor comprising a CAD-glucokinase as the glucose sensor component. The biosensor can be incorporated into a hand-held device for conventional glucose monitoring, or into an implantable device as part of an open loop system for continuous glucose monitoring. Alternatively, it can be incorporated into a closed loop biomedical device for continuous glucose monitoring and insulin delivery. One skilled in the art will understand that a closed loop system can consist of a single unit comprising the biosensor and the insulin delivery system, or the biosensor and the insulin delivery system may constitute separate units. Advantages of separate units include optimal positioning of each unit, for example, the insulin delivery unit in the portal system and the glucose-sensing unit subcutaneously to facilitate access. The two units can be connected, for example, via a short telecommunications system utilising appropriate algorithms to dictate insulin delivery.

It will be readily understood by one skilled in the art that the CAD-glucokinase according to the present invention can be incorporated into various biosensor formats for use as a glucose sensor, including those devices described above and elsewhere. The field of biosensors and bioelectronic devices is rapidly evolving and new types of these devices are continuously being developed. The use of the CAD-glucokinase as a glucose sensor in both known and newly developed devices is therefore considered to be within the scope of the present invention.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

To gain a better understanding of the invention described herein, the following examples are set forth. It should be understood that these examples are for illustrative purposes only. Therefore, they should not limit the scope of this invention in any way.

### EXAMPLES

### Example 1

### Cloning Human Glucokinase

The human liver glucokinase was cloned from the Hep 3B liver cell line. Following isolation of total mRNA from the cell line using standard techniques, RT-PCR was employed to generate sufficient glucokinase cDNA. Expand reverse transcriptase, a genetically engineered version of MoMuLV-RT that has negative RNase H activity, and an Oligo (dT)15 primer were used for the reverse transcription step. Pwo DNA Polymerase was used for the PCR step. PCR was performed in three separate reactions. The first reaction amplified a 5′ portion of the glucokinase cDNA, the second reaction amplified a 3′ portion of the glucokinase cDNA and the third reaction amplified the complete glucokinase sequence from the combined products of the first and second reactions. Primers were used that incorporated convenient restriction enzyme sites to facilitate cloning into appropriate vectors. Primers used to amplify the glucokinase for cloning into plasmid pcDNA3 (digested with BamHI and EcoRI) were:

| | |
|---|---|
| LGK-2 | |
| 5′-CCGGATCCAGATGGCGATGGATGTCACA-3′ | [SEQ ID NO:3] |
| SAC Ib | |
| 5′-GGTTTGCAGAGCTCTCGTCCAC-3′ | [SEQ ID NO:4] |
| SAC Ia | |
| 5′-GTGGACGAGAGCTCTGCAAACC-3′ | [SEQ ID NO:5] |
| GLK-3 | |
| 5′-CTGAATTCACTGGCCCAGCATACAG-3′ | [SEQ ID NO:6] |

Primers used to amplify. the glucokinase for cloning into plasmid pGEX-KG (digested with Xho I and Hind III) were:

| | |
|---|---|
| LGK-3 | |
| 5′-CCCTCGAGATGGCGATGGATGTCACA-3′ | [SEQ ID NO:7] |
| SAC Ib | |
| 5′-GGTTTGCAGAGCTCTCGTCCAC-3′ | [SEQ ID NO:4] |
| SAC Ia | |
| 5′-GTGGACGAGAGCTCTGCAAACC-3′ | [SEQ ID NO:5] |
| GLK-3-2 | |
| 5′-CTAAGCTTACTTGGCCCAGCATACAG-3′ | [SEQ ID NO:8] |

The final PCR products were cloned into pcDNA3 and pGEX-KG plasmids digested with the restriction enzymes indicated above and the inserts were sequenced.

The pGEX-KG glucokinase clone 20 nucleotide sequence was confirmed to be the same as the wild-type sequence (i.e. the protein coding region of the human major liver glucokinase cDNA, corresponding to nucleotides 160 to 1569 of GenBank Accession No. NM_{—}033507; SEQ ID NO:1). The glucokinase coding sequence from this clone was subcloned (using BamHI and HindlIl restriction sites) into pcDNA3 (BamHI/EcoRV digested) to give pcDNA3 glucokinase clone 20. The glucokinase nucleotide sequence in both plasmid pGEX-KG glucokinase clone 20 and plasmid pcDNA3 glucokinase clone 20 is identical to the protein coding region of the published sequence of the human major liver glucokinase cDNA, corresponding to nucleotides 169 to 1569 of GenBank Accession Number NM_033507; SEQ ID NO: 1.

### Example 2

### Site-Directed Mutagenesis of the Cloned Human Glucokinase

In vitro site-directed mutagenesis of the glucokinase was achieved by PCR-based techniques to create mutations at position 336 (Ser->Val; Ser->Leu and Ser->Ile) and at position 205 (Asp->Ala). The PCR reactions employed complementary primers containing mutagenic sequences, and a set of upstream and downstream primers. The sequences of the mutagenic primers were as follows (nucleotides that are different from those that occur in the wild type sequence are underlined):

| | |
|---|---|
| Ser336Val | |
| Primer A | |
| 5′-TCGTGGTCCAGGTGGAGAGCG-3′ | [SEQ ID NO:9] |
| Primer B | |
| 5′-CGCTCTCCACCTGGACCACGA-3′ | [SEQ ID NO:10] |
| Ser336Leu | |
| Primer A | |
| 5′-TCGTGCTGCAGGTGGAGAGCG-3′ | [SEQ ID NO:11] |
| Primer B | |
| 5′-CGCTCTCCACCTGCAGCACGA-3′ | [SEQ ID NO:12] |
| Ser336Ile | |
| Primer A | |
| 5′-TCGTGATTCAGGTGGAGAGCG-3′ | [SEQ ID NO:13] |
| Primer B | |
| 5′-CGCTCTCCACCTGAATCACGA-3′ | [SEQ ID NO:14] |
| Asp205Ala | |
| Primer A | |
| 5′-GGTGAATGCAACGGTGGCCACG-3′ | [SEQ ID NO:15] |
| Primer B | |
| 5′-CGTGGCCACCGTTGCATTCACCC-3′ | [SEQ ID NO:16] |
| Primer GLK-3 | |
| 5′-CTGAATTCACTGGCCCAGCATACAG-3′ | [SEQ ID NO:6] |
| Primer 4A | |
| 5′-GACTTCCTGGACAAGCATCAGA-3′ | [SEQ ID NO:17] |

PCR products with overlapping sequences in which lie the implanted missense mutations were generated by three PCR reactions. All PCR reactions were performed using Vent DNA polymerase. For each of the Ser336 and Asp 205 mutants:

PCR Reaction 1) Primer 4A (upstream primer) and Primer B;

PCR Reaction 2) Primer A and Primer GLK-3 (downstream primer); and

PCR Reaction 3) mixture of products of PCR Reactions 1 and 2 with Primer 4A and Primer GLK-3.

The final PCR product for each mutant was digested with SacII and BsrGI and re-introduced into the pcDNA3 glucokinase clone (digested with SacII and BsrGI).

### Example 3

### Generation of Wild-Type and Mutant Glucokinase Vectors

The Ser336 and Asp205 mutant glucokinases produced by the above PCR reactions were first cloned into pcDNA3 (as indicated above). Wild-type glucokinase and the mutant glucokinases were each subsequently subcloned into the pGEX-KG and pET-15b expression vectors using Xhol and BamHI restriction enzymes (blunt end) for the wild-type and Sac II and BsrGI for the mutants. The pGEX-KG and pEt-15b vectors were used in order to express wild-type and mutant glucokinases containing GST and polyhistidine (His) affinity tags, respectively.

The following plasmids were generated in this manner. All plasmids have been sequenced to confirm the presence of the appropriate mutant sequence and the absence of any abnormalities.

TABLE 1
List of Plasmids

| Plasmid | Clone # | Glucokinase |
|---|---|---|
| pGEX-KG | 20 | Wild-type |
| | 23 | Ser336Val |
| | 32 | Ser336Leu |
| | 43 | Ser336Ile |
| | 53 | Asp205Ala |
| pCDNA3 | 20 | Wild-type |
| | 7 | Ser336Val |
| | 15 | Ser336Leu |
| | 25 | Ser336Ile |
| | 33 | Asp205Ala |
| pET-15b | 14 | Wild-type |
| | 1 | Ser336Val |
| | 9 | Ser336Leu |
| | 13 | Ser336Ile |
| | 18 | Asp205Ala |

### Example 4

### Expression and Analysis of Wild-Type and Mutant Glucokinases

As described above, glucokinase was cloned and several missense mutations introduced by PCR site-directed mutagenesis. These were chosen on the basis of available information derived from X-ray crystallography modeling and from naturally occurring human mutations [loss of function mutations in MODY (maturity-onset of diabetes of the young) and gain of function mutations in neonatal hypoglycemia]. The re-engineered proteins were designed to possess null enzymatic activity while retaining performance as a pure glucose binder with all with inherent characteristics essential for biosensing. Different re-engineered mutant proteins have different glucose affinities; therefore specific glucose biosensing capabilities can be “dialed in”. In essence, synthetic glucose receptors were created.

All re-engineered glucokinase candidates were introduced into a high-level protein expression system and further modified to allow quick and easy purification by employing Ni²⁺ metal resin column chromatography. In the final analysis, recognition of bound glucose relies on the well-described large conformational change that glucokinase undergoes when glucose binds, which can be verified by fluorescent spectroscopy studies.

To allow glucokinase to perform as a simple glucose sensor, several alterations were introduced into the protein, as discussed above. The first genetic re-engineering goal was to deprive glucokinase of its enzymatic activity (Asp205Ala) without affecting glucose binding. This has the effect of simplifying binding properties since potential enzymatic sequelae are eliminated.

Other genetics changes were made to effectively abolish the ATP-binding site. There is a potential, in view of introducing a null enzymatic phenotype, that ATP binding would or could create a temary complex that would be unable to react to changes in glucose concentration in real time. The ATP-binding site was abolished by targeting an amino acid interacting with ATP as far removed from the glucose binding site as possible [Mahalingam B. et al Diabetes 48:1698-1705, 1999]; Ser336Leu, Ser336Val, Ser336Ile.

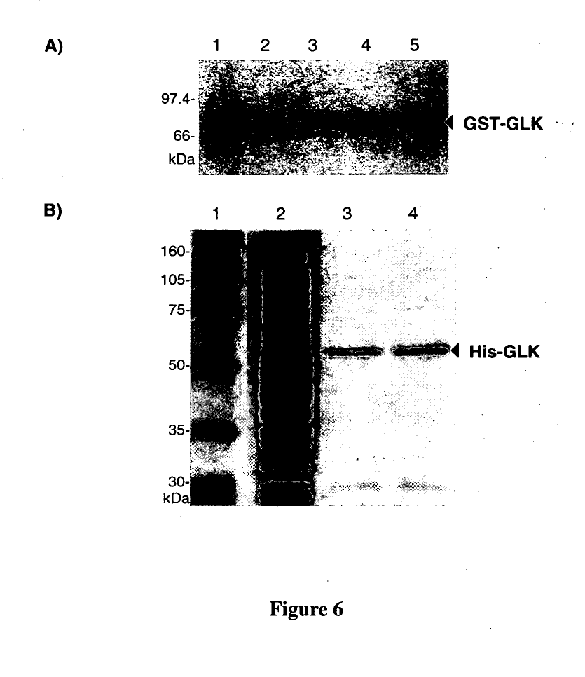

All bacterial expression constructs expressed wild-type and mutant glucokinase proteins in large quantities and the mutant GST-GLKs appear as stable as the wild-type GST-glucokinase fusion protein (FIG. 6A). Ni²⁺ affinity purification produced large quantities of >95% pure His-GLK using a simple procedure with a yield of 100 μg of protein/250 ml bacterial culture (FIG. 6B). Wild-type His-GLK eluted from the Ni²⁺ affinity column exhibited the expected enzymatic activity as described below.

### Example 5

### Analysis of Wild-Type and Mutant Glucokinase Catalytic Activity

i) Analysis of Catalytic Activity

Glucokinase activity was assayed as described previously [Storer, A. C., et al., Biochem. J. 141:205-209 (1974)] with the following modifications. Reactions were carried out at 25° C. in 50 nM glycylglycinate buffer, pH 7.8, containing 1 mM NADP, 100 mM KCl, 5 mM MgCl₂, 1 unit of glucose 6-P dehydrogenase, 100 mM glucose, 5 mM ATP, and glucokinase in a total volume of 1 ml. All reaction mixtures were incubated at 25° C.

Production of NADPH was followed by the increase in absorbance at 340 nm using a spectrophotometer. Glucokinase activity may then be calculated using the following formula:
Activity (units/ml)=(Δ OD/min)/0.15×dilution factor

A unit of activity is the amount of glucokinase which transforms, under optimal conditions, 1 μmole of substrate/min at room temperature.

In vitro site-directed mutagenesis of glucokinase was achieved by using PCR-based techniques to create mutations at position 205 (Asp205Ala) and at position 336 (Ser>Val; Ser>Leu and Ser>Ile) as described abvoe. The final PCR product for each mutant was digested with appropriate restriction enzymes and re-introduced into the wild-type pcDNA3-GLK clone. Each mutant pcDNA3 clone was transfected into COS-1 cells using standard liposomal transfection methodology. On day 3 post transfection, glucokinase activity was measured in COS-1 cell extracts (as described in Trifiro, M. & Nathan, D., Prep. Biochem. 16:155-173, 1986) and all mutant glucokinase proteins were shown to have null enzyme activity (i.e. below detectable limits). pcDNA3-wt GLK-transfected cell extracts displayed significant measurable glucokinase enzyme activity.

Wild-type and mutant glucokinases (Asp205Ala, Ser336Val, Ser336Leu, Ser336Ile) were subsequently subcloned into the pGEX-KG (GST-GLK) and pET-15b (His-GLK) bacterial expression vectors using appropriate restriction enzymes. These plasmids introduce a GST tag or polyhistidine metal affinity tag to the N-terminus of glucokinase, allowing for one-step purification of glucokinase from bacterial lysates using glutathione or Ni²⁺columns. The glucokinase activity of the purified glucokinases was assayed as above. Both wild-type GST-GLK and His-GLK had significant glucokinase enzymatic activity, demonstrating that adding an N-terminal affinity tag did not impede catalytic activity. The purified mutant His-GLK proteins (Asp205Ala, Ser336Val, Ser33Leu, Ser336Ile) had less than 1% of the glucokinase activity of the wild-type His-tagged glucokinase, confirming that these mutant glucokinases had null enzyme activity.

The enzymatic activity of the wild-type GST and His-tagged glucokinases was measured while the proteins were immobilized on glutathione or Ni²⁺-containing supports, respectively. Both GST-GLK and His-GLK retained glucokinase activity, demonstrating that immobilization on a solid support did not impede enzymatic activity.

### Example 6

### Conformational Studies using Intrinsic Fluorescence Spectroscopy

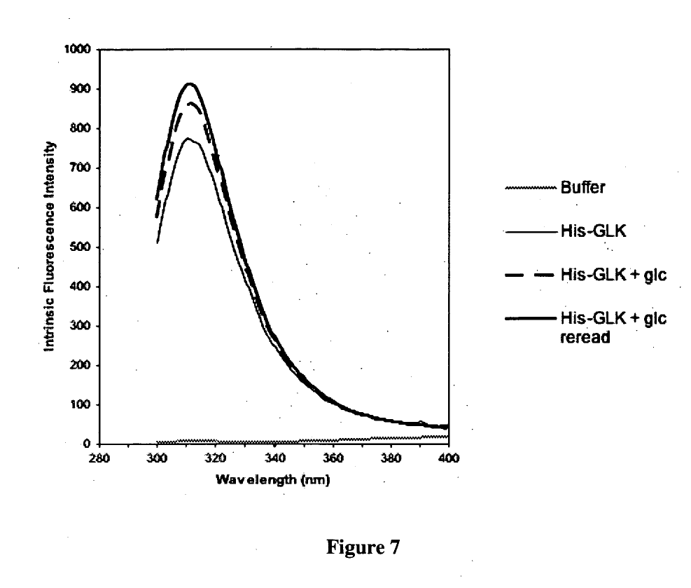

Intrinsic fluorescence experiments with glucokinase were performed to document and verify glucose-induced conformational changes, which is an absolute requirement for impedance biosensing to be successful. Purified wild-type N-terminal His-tagged glucokinase (10 μg) with enzyme specific activity of ≅200 U/mg was used in a 10×10 mm² cuvette. After the addition of 100 mM glucose, intrinsic fluorescence spectroscopy was performed (excitation wavelength 280 nm; maximum fluorescence 312 nm). There is a 20-25% increase in maximal intrinsic fluorescence at 312 nm with the addition of 100 mM glucose, reflective of conformational changes in the His-tagged glucokinase (FIG. 7). This is similar to native wild-type glucokinase [Xu L Z, Zhang W, Weber I T, Harrison R W, Pilkis S J. 1994. Site-directed mutagenesis studies on the determinants of sugar specificity and cooperative behavior of human beta-cell glucokinase. J Biol Chem 269:27458-27465; and Xu L Z, Weber I T, Harrison R W, Gidh-Jain M, Pilkis S J. 1995. Sugar specificity of human beta-cell glucokinase: correlation of molecular models with kinetic measurements. Biochemistry 34:6083-6092], which leads to the conclusion that N-terminal manipulation of glucokinase does not introduce any untoward effects on the enzyme.

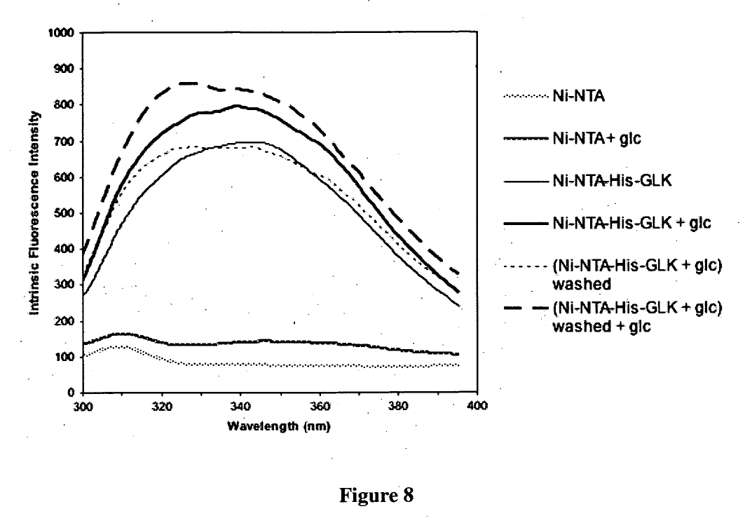

Intrinsic fluorescence experiments were performed on purified immobilized wild-type N-terminal His-tagged glucokinase (FIG. 8). There was an increase in the maximal intrinsic fluorescence of the Ni²⁺-bound His-GLK in the presence of 100 mM glucose compared to the Ni²⁺-bound His-GLK in the absence of glucose, suggesting that glucokinase can undergo a conformational shift when bound to a solid support. Next, immobilized His-GLK that had been previously incubated with glucose was washed with glucose-free buffer. The maximal intrinsic fluorescence was similar to that of the Ni²⁺-bound His-GLK, suggesting that glucose binding is reversible. When the washed Ni²⁺-bound His-GLK was incubated with glucose, again an upward shift in the fluorescence intensity occurred, indicative of a recurring conformational change in the protein.

### Example 6

### Creation of a Reagentless Biosensor

To establish the use of modified re-engineered glucokinase molecules as potential mediators of glucose recognition, experiments were performed using impedance biosensing technology. The initial objectives were to covalently immobilize wild-type glucokinase on two potential electrode substrates: silicon (Si; industry standard), and screen-printed graphite (SP) electrodes [Marquette, C. A. et al., Anal. Chem., 78:959-964 (2006)]. Impedance measurements were used to monitor the changes of glucokinase in the presence of glucose. Wild-type glucokinase was chosen due to the ease of monitoring enzymatic activity at every step of immobilization. Loss of enzymatic activity would most likely reflect sufficient damage to the protein and preclude any chance that the wild-type/mutant glucokinase could function as a biosensor. These prototype sensors displayed a very reproducible shift in impedance measurements in the presence of D-glucose, on a surface area of only 780 μm².

Initial experiments using the accepted practice of nitrobenzenediazoniun/glutaraldehyde coupling of substances to electrode surfaces did not lead to a functional glucose sensor. Further experiments showed that sufficient glucokinase was indeed covalently coupled to the surface, but the process itself leads to a total loss of enzyme activity. This was not a complete surprise, as the final coupling requires free amino groups of the protein. Most likely there are a significant number of such groups involved, but in the process of participating in the coupling, enzyme function is destroyed.

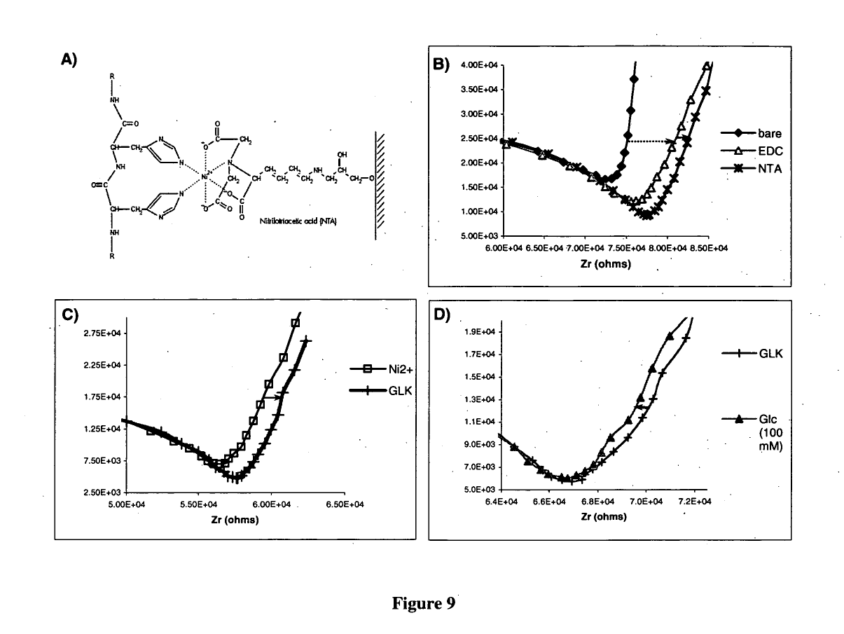

Since it had previously been shown that Ni²⁺-immobilized His-tagged glucokinase retained full enzymatic activity a similar strategy was used wherein Ni²⁺ was fixed on the surface of the electrode, and then His-GLK was attached. Each step was analyzed by bioimpedance measurements. First, nitrilotriacetic acid (NTA) ligand (FIG. 9A) was coupled to SP electrode surface using 1-ethyl-3-(3-dimethylaminopropyl)-carbodiimide (EDC) activation (FIG. 9B). Electrodes were then loaded with Ni²⁺ cations. Wild-type His-GLK (1 μg) was then avidly coupled to the electrode surface, again corroborated by impedance measurements (FIG. 9C). These prepared electrodes were subsequently used as glucose biosensors. All displayed significant impedance changes in the presence of 100 mM glucose (FIG. 9D).

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. An isolated or purified recombinant human glucokinase polypeptide having decreased catalytic activity but a substantially identical ability to bind glucose relative to a corresponding wild-type human glucokinase. polypeptide, wherein
said corresponding wild-type human glucokinase polypeptide has an amino acid sequence as set forth in GenBank Accession No. AAA52562, GenBank Accession No. AAA51824, GenBank Accession No. NP_{—}000153, GenBank Accession No. P35557; GenBank Accession No. AAB97681, GenBank Accession No. NP_{—}277042; GenBank Accession No. AAB97682, GenBank Accession No. NP_{—}277043; GenBank Accession No. AAB59563 or SEQ ID NO:2, or is encoded by a nucleotide sequence as set forth in GenBank Accession No. M90299, GenBank Accession No. M88011, GenBank Accession No. NM_{—}000162, GenBank Accession No. NM_{—}033507; GenBank Accession No. NM_{—}033508; GenBank Accession No. M69051, GenBank Accession No. AH005826 or SEQ ID NO:1;
said recombinant human glucokinase polypeptide comprises an amino acid sequence that differs from the sequence of the corresponding wild-type human glucokinase polypeptide by a mutation at an amino acid residue corresponding to Asp 78, Ser 151, Asp 205, Arg 85, Lys 169, Gly 81, Lys 169, Thr 82, Asn83, Thr228, Ser 411, Lys 296, Ser 336, Thr332, Glu 290, Thr268, Glu256, Asn204, Asn231 or Lys269 of said corresponding wild-type human glucokinase polypeptide; and
said recombinant human glucokinase polypeptide further comprises at least one affinity tag or reactive group for immobilizing said recombinant human glucokinase polypeptide onto a solid surface.

2. The isolated or purified recombinant human glucokinase polypeptide of claim 1, wherein said recombinant human glucokinase polypeptide has the amino acid sequence of SEQ ID NO:2 or is encoded by the nucleotide sequence of SEQ ID NO:1.

3. The isolated or purified recombinant human glucokinase polypeptide of claim 1, wherein said affinity tag or reactive group for immobilizing said recombinant human glucokinase polypeptide onto a solid surface is a metal-binding motif or an affinity tag.

4. The isolated or purified recombinant human glucokinase polypeptide of claim 3, wherein affinity tag or reactive group for immobilizing said recombinant human glucokinase polypeptide onto a solid surface is a glutathione-S-transferase (GST) tag, a polyhistidine tag, avidin, streptavidin or biotin.

5. The isolated or purified recombinant human glucokinase polypeptide of claim 4, wherein affinity tag or reactive group for immobilizing said recombinant human glucokinase polypeptide onto a solid surface is a hexa-histidine tag.

6. The isolated or purified recombinant human glucokinase polypeptide of claim 1, wherein said solid surface is part of a biosensor.

7. The isolated or purified recombinant human glucokinase polypeptide of claim 1, wherein said decreased catalytic activity is between about 10 and about 10 000-fold less than the catalytic activity of the corresponding wild-type human glucokinase polypeptide.

8. An isolated or purified recombinant human glucokinase polypeptide having decreased catalytic activity but a substantially identical ability to bind glucose relative to a corresponding wild-type human glucokinase polypeptide, wherein:
said corresponding wild-type human glucokinase polypeptide has an amino acid sequence as set forth in GenBank Accession No. AAA52562, GenBank Accession No. AAA51824, GenBank Accession No. NP_{—}000153, GenBank Accession No. P35557; GenBank Accession No. AAB97681, GenBank Accession No. NP_{—}277042; GenBank Accession No. AAB97682, GenBank Accession No. NP_{—}277043; GenBank Accession No. AAB59563 or SEQ ID NO:2, or is encoded by a nucleotide sequence as set forth in GenBank Accession No. M90299, GenBank Accession No. M88011, GenBank Accession No. NM_{—}000162, GenBank Accession No. NM_{—}033507, GenBank Accession No. NM_{—}033508; GenBank Accession No. M69051; GenBank Accession No. AH005826 or SEQ ID NO:1;
said recombinant human glucokinase polypeptide comprises an amino acid sequence that differs from the sequence of the corresponding wild-type human glucokinase polypeptide by a mutation at the amino acid residue corresponding to Asp 205 of said corresponding wild-type human glucokinase polypeptide; and
said recombinant human glucokinase polypeptide further comprises at least one affinity tag or reactive group for immobilizing said recombinant human glucokinase polypeptide onto a solid surface.

9. The isolated or purified recombinant human glucokinase polypeptide of claim 8, wherein said recombinant human glucokinase polypeptide has the amino acid sequence of SEQ ID NO:2 or is encoded by the nucleotide sequence of SEQ ID NO:1.

10. The isolated or purified recombinant human glucokinase polypeptide of claim 8 wherein said mutation is Asp205Ala.

11. The isolated or purified recombinant human glucokinase polypeptide of claim 8, wherein affinity tag or reactive group for immobilizing said recombinant human glucokinase polypeptide onto a solid surface is a metal-binding motif or an affinity tag.

12. The isolated or purified recombinant human glucokinase polypeptide of claim 11, wherein affinity tag or reactive group for immobilizing said recombinant human glucokinase polypeptide onto a solid surface is a glutathione-S-transferase (GST) tag, a polyhistidine tag, avidin, streptavidin or biotin.

13. The isolated or purified recombinant human glucokinase polypeptide of claim 12, wherein affinity tag or reactive group for immobilizing said recombinant human glucokinase polypeptide onto a solid surface is a hexa-histidine tag.

14. The isolated or purified recombinant human glucokinase polypeptide of claim 1, wherein:
said recombinant human glucokinase polypeptide has the amino acid sequence of SEQ ID NO:2 or is encoded by the nucleotide sequence of SEQ ID NO:1;
said mutation is Asp205Ala; and
affinity tag or reactive group tag for immobilizing said recombinant human glucokinase polypeptide onto a solid surface is a glutathione-S-transferase (GST) tag or a polyhistidine tag.

15. The isolated or purified recombinant human glucokinase polypeptide of claim 8, wherein said solid surface is part of a biosensor.

16. The isolated or purified recombinant human glucokinase polypeptide of claim 14, wherein said solid surface is part of a biosensor.

17. The isolated or purified recombinant human glucokinase polypeptide of claim 8, wherein said decreased catalytic activity is between about 10 and about 10 000-fold less than the catalytic activity of the corresponding wild-type human glucokinase polypeptide.

18. An isolated or purified recombinant human glucokinase polypeptide having decreased catalytic activity but a substantially identical ability to bind glucose relative to a corresponding wild-type human glucokinase. polypeptide, wherein:
said corresponding wild-type human glucokinase polypeptide has an amino acid sequence as set forth in GenBank Accession No. AAA52562, GenBank Accession No. AAA51824, GenBank Accession No. NP_{—}000153, GenBank Accession No. P35557; GenBank Accession No. AAB97681, GenBank Accession No. NP_{—}277042; GenBank Accession No. AAB97682, GenBank Accession No. NP_{—}277043; GenBank Accession No. AAB59563 or SEQ ID NO:2, or is encoded by a nucleotide sequence as set forth in GenBank Accession No. M90299, GenBank Accession No. M88011, GenBank Accession No. NM_{—}000162, GenBank Accession No. NM_{—}033507; GenBank Accession No. NM_{—}033508; GenBank Accession No. M69051, GenBank Accession No. AH005826 or SEQ ID NO:1;
said recombinant human glucokinase polypeptide comprises an amino acid sequence that differs from the sequence of the corresponding wild-type human glucokinase polypeptide by a mutation at the amino acid residue corresponding to Ser336 of said corresponding wild-type human glucokinase polypeptide; and
said recombinant human glucokinase polypeptide further comprises at least one affinity tag or reactive group for immobilizing said recombinant human glucokinase polypeptide onto a solid surface.

19. The isolated or purified recombinant human glucokinase polypeptide of claim 18, wherein said recombinant human glucokinase polypeptide has the amino acid sequence of SEQ ID NO:2 or is encoded by the nucleotide sequence of SEQ ID NO:1.

20. The isolated or purified recombinant human glucokinase polypeptide of claim 18 wherein said mutation is Ser336Val, Ser336Leu or Ser336Ile.

21. The isolated or purified recombinant human glucokinase polypeptide of claim 18, wherein affinity tag or reactive group for immobilizing said recombinant human glucokinase polypeptide onto a solid surface is a metal-binding motif or an affinity tag.

22. The isolated or purified recombinant human glucokinase polypeptide of claim 21, wherein affinity tag or reactive group for immobilizing said recombinant human glucokinase. polypeptide onto a solid surface is a glutathione-S-transferase (GST) tag, a polyhistidine tag, avidin, streptavidin or biotin.

23. The isolated or purified recombinant human glucokinase polypeptide of claim 22, wherein affinity tag or reactive group for immobilizing said recombinant human glucokinase polypeptide onto a solid surface is a hexa-histidine tag.

24. The isolated or purified recombinant human glucokinase polypeptide of claim 18, wherein:
said recombinant human glucokinase polypeptide has the amino acid sequence of SEQ ID NO:2 or is encoded by the nucleotide sequence of SEQ ID NO:1;
said mutation is Ser336Val, Ser336Leu or Ser336Ile; and
said affinity tag or reactive group for immobilizing said recombinant human glucokinase polypeptide onto a solid surface is a glutathione-S-transferase (GST) tag or a polyhistidine tag.

25. The isolated or purified recombinant human glucokinase polypeptide of claim 18, wherein said solid surface is part of a biosensor.

26. The isolated or purified recombinant human glucokinase polypeptide of claim 24, wherein said solid surface is part of a biosensor.

27. The isolated or purified recombinant human glucokinase polypeptide of claim 18, wherein said decreased catalytic activity is between about 10 and about 10 000-fold less than the catalytic activity of the corresponding wild-type human glucokinase polypeptide.
